(19) United States
(12) Patent Application Publication
(10) Pub. No.: US 2002/0132802 A1
(43) Pub. Date: Sep. 19, 2002

(54) Cytoprotective polycyclic compounds

(75) Inventor: Douglas F. Covey, Ballwin, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Washington Univeristy

(21) Appl. No.: 10/008,567

(22) Filed: Nov. 5, 2001

Related U.S. Application Data
(60) Provisional application No. 60/249,580, filed on Nov. 17, 2000.

(51) Int. Cl. A61K 31/56

(52) U.S. Cl. 514/178; 514/182

(57) ABSTRACT

The present invention is generally directed to novel enantiomeric estrogen derivatives, some of which may have one or more unsaturated bonds in conjugation with the terminal or A-ring of the structure, which have cytoprotective activity. The present invention is further directed to a process for conferring cytoprotection to a population of cells, of a subject in need thereof, involving the administration of an effective dose of the compound.

### REFERENCE TO RELATED APPLICATION

This application claims priority from U.S. provisional application, Ser. No. 60/249,580, filed on Nov. 17, 2000, the entire contents of which is incorporated herein by reference.

### BACKGROUND OF THE INVENTION

The present invention is generally directed to novel compounds with cytoprotective activity, and the uses thereof, the compounds having a polycyclic structure with a terminal hydroxy-substituted or hydroxy-bearing aromatic ring, the structure optionally containing one or more unsaturated bonds in conjugation therewith. More specifically, the present invention is directed to novel enantiomeric estrogen derivatives, some of which may have one or more unsaturated bonds in conjugation with the terminal or A-ring of the structure. The present invention is further directed to a process wherein cytoprotective activity is conferred to a population of cells by the administration of such a compound.

There continues to be a need for treatments that protect cells from cell death resulting from episodes of, for example, disease, trauma, isolation and removal of tissues or cells from the body, or exposure to toxins. This need extends to, among other things: (i) treatments for nerve cell loss associated with chronic or acute neurodegenerative disorders or trauma; (ii) treatments to minimize tissue damage resulting from ischemia where ischemia may occur as a result of stroke, heart disease, a transplantation event, or other event resulting in a cut-off in nutritional supply to tissues; and, (iii) treatments to modulate cell death associated with other degenerative conditions (such as osteoporosis or anemia). The absence of an effective cytoprotective therapy can result in either loss of life or a general decline in the quality of life, including permanent disability with high health care costs to patients, their families and the health care providers.

One approach to minimizing such pathologic changes has been to attempt to neutralize the oxidative stress or damage that is associated with the accumulation of free radicals in the extracellular space when these changes occur. For example, Mooradian has reported that certain estrogens have significant anti-oxidant properties in in vitro biochemical assays, but that this effect is not seen with all estrogens. (See, J. Steroid Biochem. Molec. Biol., 45 (1993) 509-511.)

Because of the variation in anti-oxidant properties noted by Mooradian in his biochemical assays, he concluded steroid molecules must have certain anti-oxidant determinants which were as yet unknown. Similar observations concerning steroids with phenolic A rings were reported in PCT Patent Application No. WO 95/13076, wherein biochemical assays were used to show anti-oxidant activity. However, the assays used by Mooradian, as well as those used in WO 95/13076, were biochemical assays and, as such, did not directly examine the effects of these molecules on cells. In contrast, Simpkins et al. describe, in U.S. Pat. No. 5,554,601 for example, cell-based assays to determine a method of conferring neuroprotection on a population of cells using estrogen compounds based on demonstrated cell protective effects. As a result, in recent years it has become recognized that estrogen, as well as other polycyclic phenols, may be used for this purpose. (See, e.g., U.S. Pat. Nos. 5,972,923; 5,877,169; 5,859,001; 5,843,934; 5,824,672; and, 5,554,601; all of which are incorporated herein by reference.)

The mechanism by which estrogen compounds bring about a neuroprotective effect is still not fully understood. However, these compounds have been shown to have a number of different physiological and biochemical effects on neurons. For example, estrogen has been shown to stimulate the production of neurotrophic agents that in turn stimulate neuronal growth. Estrogen compounds have also been found to inhibit NMDA-induced cell death in primary neuronal cultures (see, e.g., Behl et al. Biochem. Biophys Res. Commun. (1995) 216:973; Goodman et al. J. Neurochem. (1996) 66:1836), and further to be capable of removing oxygen free radicals and inhibiting lipid peroxidation (see, e.g., Droescher et al. WO 95/13076). While the potential effect of free radicals on neurons per se is unproven, as noted above it is currently believed that the ability to scavenge free radicals is one desirable property and, as a result, is something that many have further examined. For example, Droeschler et al. describe cell free in vitro assay systems using lipid peroxidation as an endpoint in which several estrogens, as well as vitamin E, were shown to have activity. Estradiol has also been reported to reduce lipid peroxidation of membranes (see, e.g., Niki (1987) Chem. Phys. Lipids 44:227; Nakano et al. Biochem. Biophys. Res. Comm. (1987) 142:919; Hall et al. J. Cer. Blood Flow Metab. (1991)11:292). Other compounds, including certain 21-amino steroids and a glucocorticosteroid, have been found to act as anti-oxidants and have been examined for their use in spinal cord injury, as well as head trauma, ischemia and stroke. (See, e.g., Wilson et al. (1995) J. Trauma 39:473; Levitt et al. (1994) J. Cardiovasc. Pharmacol 23:136; Akhter et al. (1994) Stroke 25; 418).

While anti-oxidant behavior is believed to be an important property, a number of other factors are believed to be involved in achieving neuroprotection. As a result, it is to be noted that therapeutic agents selected on the basis of a single biochemical mechanism may have limited generalized utility in treating disease or trauma in patients. For example, in order to achieve an anti-oxidant effect in vitro using estrogen, Droescher et al. used very high doses of estrogens. Such doses, even if effective on neurons in vivo, would have limited utility in treating chronic neurological conditions because of associated problems of toxicity that result from the prolonged use of these high dosages.

In addition to the issues related to compound toxicity, consideration must also be given to the ability of a particular compound to reach the target site, which in some applications is controlled by the ability of the compound to cross the blood-brain barrier. The blood-brain barrier is a complex of morphological and enzymatic components that retards the passage of both large and small charged molecules, and thus limits the access of such molecules to cells of the brain. Furthermore, not only must the compound be capable of reaching the target site, but it must also do so in a state or configuration which enables it to carry-out its designated function.

In view of the foregoing, it can be seen that a need continues to exist for the identification of compounds which have demonstrated biological efficacy in protecting humans from the consequences of abnormal cell death in body tissue; compounds which are capable of crossing the blood-brain barrier and which are suitable for administration in dosages which are non-toxic. This identification requires continuing advances in the understanding of the structural requirements for compositions capable of inducing neuroprotection, which in turn provide the basis for designing novel drugs that have enhanced cytoprotective properties while at the same time have reduced adverse side effects.

### SUMMARY OF THE INVENTION

Among the several objects and features of the present invention include the provision of a compound having cytoprotective activity which is an enantiomeric estrogen derivative; the provision of such a compound having neuroprotective activity; and, the provision of such a compound wherein one or more unsaturated bonds are present and in conjugation with the terminal, hydroxy-substituted aromatic ring (i.e., the A-ring).

Further among the objects and features of the invention is the provision of a compound which is an estrogen derivative and which has cytoprotective activity, wherein the D-ring has a spiro substituent bound thereto; the provision of such a compound having neuroprotective activity; and, the provision of such a compound which optionally has the enantiomeric configuration of the naturally-occurring analog thereof.

Still further among the objects and features of the invention is the provision of a process for treating a population of cells against cell death or cell damage wherein an effective dose of such a compound as described above is administered thereto.

Briefly, therefore, the present invention is directed to a process for treating a cytodegenerative or neurodegenerative disease comprising administering to a subject in need thereof a compound having formula (I), or one of the various diastereomer thereof:

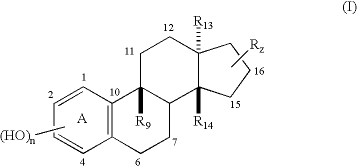

wherein

the compound optionally has one or more unsaturated bonds in conjugation with the aromatic A-ring between carbons 6 and 7, 8 and 9, or 9 and 11, in which event one or both of R⁸ and R⁹ will be absent;

n ranges from 1 to 4;

R⁸ and R⁹, when present, are independently hydrogen or alkyl;

R¹³ is hydrogen, substituted or unsubstituted hydrocarbyl, halo, amido, sulfate or nitrate;

R¹⁴ is hydrogen or alkyl;

R^{z} is hydrogen, hydroxy, oxo, substituted or unsubstituted hydrocarbyl, heterocycloalkyl, heterocycloalkenyl, halo, amido, sulfate, or nitrate; and,

carbon 17 and carbon 3 are not each hydroxy-substituted when (i) n is 1, (ii) the compound does not contain at least one unsaturated bond in conjugation with the aromatic A-ring, (iii) R⁸, R⁹ and R¹⁴ are hydrogen, and (iv) R¹³ is methyl.

The present invention is further directed to a process for treating a cytodegenerative or neurodegenerative disease comprising administering to an individual in need thereof a compound having formula (II), or a stereoisomer thereof:

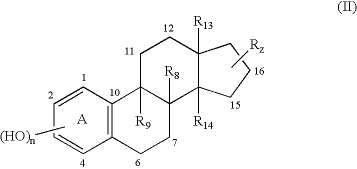

wherein

the compound optionally has one or more unsaturated bonds in conjugation with the aromatic A-ring between carbons 6 and 7, 8 and 9, or 9 and 11, in which event one or both of R⁸ and R⁹ will be absent;

n ranges from 1 to 4;

R⁸ and R⁹, when present, are independently hydrogen or alkyl;

R¹³ is hydrogen, substituted or unsubstituted hydrocarbyl, halo, amido, sulfate or nitrate;

R¹⁴ is hydrogen or alkyl;

R^{z} is substituted or unsubstituted cycloalkyl or cycloalkenyl, or substituted or unsubstituted heterocycloalkyl or heterocycloalkenyl.

The present invention is still further directed to a process for treating a cytodegenerative or neurodegenerative disease comprising administering to an individual in need thereof a pharmaceutical composition comprising a compound as described above.

The present invention is still further directed to a process for conferring cytoprotection or neuroprotection on a population of cells which comprises administering to that population of cells a compound as described above, or a pharmaceutical composition comprising such a compound.

The present invention is still further directed to a compound having cytoprotective activity. The compound has the formula (I), or a diastereomer thereof:

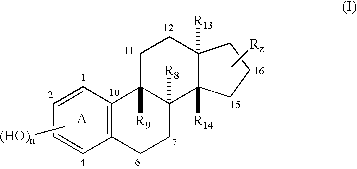

wherein

the compound optionally has one or more unsaturated bonds in conjugation with the aromatic A ring between carbons 6 and 7, 8 and 9, or 9 and 11, in which event one or both of R⁸ and R⁹ will be absent;

n ranges from 1 to 4;

R⁸ and R⁹, when present, are independently hydrogen or alkyl;

R¹³ is hydrogen, substituted or unsubstituted hydrocarbyl, halo, amido, sulfate or nitrate;

R¹⁴ is hydrogen or alkyl;

R^{z} is hydrogen, hydroxy, oxo, substituted or unsubstituted hydrocarbyl, heterocycloalkyl, heterocycloalkenyl, halo, amido, sulfate, or nitrate, provided however, when (i) the compound does not contain at least one unsaturated bond in conjugation with the aromatic A-ring, (ii) R⁸, R⁹ and R¹⁴ are hydrogen, and (iii) R¹³ is methyl, R^{z} is other than hydrogen and is not hydroxy or oxo when the D-ring is only substituted at carbon 17.

Other objects and features of the present invention will be in part apparent and in part pointed out hereinafter.

### BRIEF DESCRIPTION OF THE DRAWINGS

FIG. 1A and 1B generally illustrates chemical structures of some preferred polycyclic, hydroxy-substituted aromatic compounds as described herein, which may be used to confer cytoprotection on a population of cells upon the administration of an effective dose thereof.

FIG. 2 contains a graph which illustrates the cytoprotective activity test results of certain compounds shown, as determined by means known in the art.

FIG. 3 contains a graph which illustrates the cytoprotective activity test results of certain compounds shown, as determined by means known in the art.

FIG. 4 contains a graph which illustrates the cytoprotective activity of some preferred compounds (e.g., ZYC-10, ZYC-12, ZYC-13), as determined by means known in the art.

FIG. 5 generally illustrates chemical structures of alternatively preferred polycyclic, hydroxy-substituted aromatic compounds of the present invention, wherein Rz may be for example a hydrogen, a hydroxy group, a oxo group, or some other substituent as described herein.

FIG. 6 contains a graph which illustrates the cytoprotective activity of some alternatively preferred compounds (e.g., ZYC-2 and ZYC-4), as determined by means known in the art, wherein Rz as described above in FIG. 5 is shown here as both a hydroxy group and a second ring structure (in this case, a 5-membered, spiro ring structure).

### DETAILED DESCRIPTION OF THE PREFERRED EMBODIMENTS

It is now recognized that certain polycyclic phenolic compounds, in particular estrogen-based compounds having the general structure:

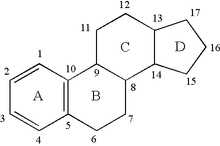

have cytoprotective, and in some cases neuroprotective, activity (see, e.g., U.S. Pat. Nos. 5,972,923; 5,877,169; 5,859,001; 5,843,934; 5,824,672; 5,554,601; 6,197,833; and, 6,207,658; all of which are incorporated herein by reference). Without being held to a particular theory, it is generally believed that the activity associated with estrogen compounds, or more generally polycyclic phenolic compounds, is, at least in part, a result of the ability of estrogens, because of their lipophilic nature, to become inserted into the cell membrane. Once in this position, the intact phenol group may donate a hydroxy hydrogen radical to prevent the cascade of membrane lipid peroxidation. Furthermore, it is generally believed that the significant potency of estrogens may result from their ability to donate a hydroxy hydrogen radical from several positions on the A-ring (see, e.g., U.S. Pat. No. 5,972,923), and because a relatively stable, oxidized form of the estrogen may result from this hydrogen radical donation (due to the effects of resonance stability).

It has now been discovered that the synthetic enantiomers of many of these compounds also possess cytoprotective activity. In particular, it has been discovered that some synthetic enantiomers of naturally-occurring steroids, such as those disclosed by Simpkins et al. (as well as dihydroxy (e.g., catechol), trihydroxy, etc. analogs of such compounds), also possess cytoprotective, and in some case neuroprotective, activity. Additionally, it has been discovered that the cytoprotective activity of these compounds may in some cases be enhanced (relative to the naturally occurring analogs thereof) when additional unsaturated bonds, which are in conjugation with the terminal aromatic ring, are present. Without being held to any particular theory, it is generally believed this additional conjugation is favorable because it allows for the formation of a more stable, oxidized form of the compound; that is, it allows for additional delocalization of the phenoxy radical, for example, which is believed to be formed as a result of the loss of a hydrogen radical to quench hydroperoxides (formed by the interaction of oxygen radical species with unsaturated fatty acids).

In this regard it should be noted that, as used herein, the prefix “Ent” refers to the enantiomer of a given compound; that is, the “Ent” designation means the orientation of chiral centers present in that compound are the opposite of those in a corresponding compound which does not have this prefix. More specifically, as used herein below, this prefix refers to the synthetic enantiomers of the corresponding naturally-occurring compounds, some of which are also shown for illustrative purposes. Generally speaking, the enantiomers disclosed herein have an absolute configuration which is opposite that of their naturally-occurring steroid counterparts (some of which are also as disclosed herein and by Simpkins et al.,) at positions C-8, C-9, C-13 and C-14. The naturally-occurring steroids have the following configurations at these positions: C-8=beta; C9=alpha; C-13=beta; and, C-14=alpha (wherein beta conventionally means the substituent extend up from, or above, the plane of the page, while conversely alpha means the substituent extend back from, or below, this plane).

Additionally, it is to be noted that the presence of a double bond between C-8 and C-9, or at between C-9 and C-11, eliminates the chiral centers in these positions.

Enantiomeric Estrogen Derivatives

In one embodiment, the present invention is directed to a process for conferring cytoprotection to a population of cells. The process comprises administering to that populations of cells a polycyclic compounds (e.g., bicyclic, tricyclic, tetracyclic, etc.) which comprise a terminal hydroxy-substituted ring, and optionally one or more unsaturated bonds which are in conjugation with the terminal ring. More specifically, the present invention is generally directed to the administration of the compound of formula (I), and/or one of the various diastereomers thereof (i.e., one of the diastereomeric configurations of the compound shown):

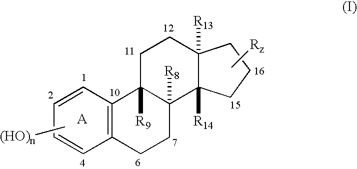

wherein, as further described and illustrated herein: the compound optionally has one or more unsaturated bonds in conjugation with the aromatic A-ring between carbons 6 and 7, 8 and 9, or 9 and 11, in which event one or both of the R⁸ and R⁹ substituents will be absent; n represents the number of hydroxy groups or substituents on the aromatic A-ring (ranging from 1 to 4, but typically being 1 or 2); R⁸ and R⁹, when present, are for example independently selected from hydrogen or substituted or unsubstituted alkyl; R¹³ is for example hydrogen, substituted or unsubstituted hydrocarbyl, halo, amido, sulfate or nitrate; R¹⁴ is for example hydrogen or alkyl; R^{z} is for example hydrogen, hydroxy, oxo, substituted or unsubstituted hydrocarbyl (e.g., alkyl, alkenyl, cycloalkyl, cycloalkenyl), heterocycloalkyl, heterocycloalkenyl, halo, amido, sulfate, or nitrate. In the present process, however, the proviso exists that carbon 17 and carbon 3 are not each hydroxy-substituted when (i) n is 1, (ii) the compound does not contain at least one unsaturated bond in conjugation with the aromatic A-ring, (iii) R⁸, R⁹ and R¹⁴ are hydrogen, and (iv) R¹³ is methyl. Stated another way, the present invention is directed to a process comprising the administration of enantiomeric estrogen derivatives other than the enantiomer of 17β-estradiol.

In another embodiment, the present invention is directed to a process for treating a cytodegenerative or neurodegenerative disease. This process comprises administering to a subject (e.g., an animal or a human) in need thereof the above-described compound.

In yet another embodiment, the present invention is directed to a compound having cytoprotective, and in some cases neuroprotective, activity. The compound has the general formula (I), or alternatively one of the various diastereomers thereof:

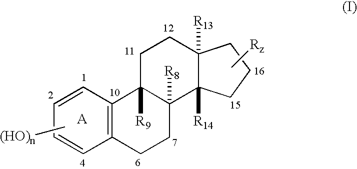

wherein, as further described and illustrated herein: the compound optionally has one or more unsaturated bonds in conjugation with the aromatic A-ring between carbons 6 and 7, 8 and 9, or 9 and 11, in which event one or both of the R⁸ and R⁹ substituents will be absent; n represents the number of hydroxy groups or substituents on the aromatic A-ring (ranging from 1 to 4, but typically being 1 or 2) ; R⁸ and R⁹, when present, are for example independently selected from hydrogen or substituted or unsubstituted alkyl; R¹³ is for example hydrogen, substituted or unsubstituted hydrocarbyl, halo, amido, sulfate or nitrate; R¹⁴ is for example hydrogen or alkyl; R^{z} is for example hydrogen, hydroxy, oxo, substituted or unsubstituted hydrocarbyl (e.g., alkyl, alkenyl, cycloalkyl, cycloalkenyl), heterocycloalkyl, heterocycloalkenyl, halo, amido, sulfate, or nitrate. In the present invention, however, the proviso applies that when (i) the compound does not contain at least one unsaturated bond in conjugation with the aromatic A-ring (e.g., an unsaturated bond between carbons 6 and 7, 8 and 9, or 9 and 11), (ii) R⁸, R⁹ and R¹⁴ are hydrogen, (iii) R¹³ is methyl, and (iv) carbon 3 is hydroxy-substituted, R^{z} is other than hydrogen and is not hydroxy or oxo when the D-ring is only substituted at carbon 17. Stated another way, the present invention is additionally directed to compounds which are enantiomeric estrogen derivatives other than the enantiomers of 17β-estradiol, estrone and estra-1,3,5(10)-trien-3-ol.

With regard to R^{z}, it is to be noted that this substituent may be attached to any of the carbon atoms which comprise the D-ring of the above structure. Typically, however, R^{z} is bound to C-16 or C-17. It is to be further noted that, in some embodiments, additional substituents may be present on the D-ring; that is, generally speaking, in some embodiments there may be more than one (e.g., 2 or 3) R^{z} substituent attached to the D-ring, the compound thus generally having the structure as shown in formula (I) or (II) herein, the D-ring appears as shown below:

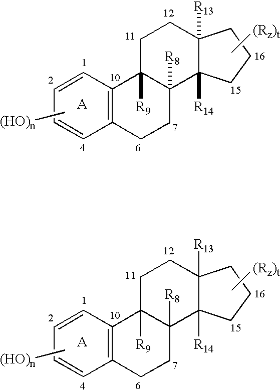

(wherein t ranges from 1 to 3). In such instances, these substituents may be the same or different.

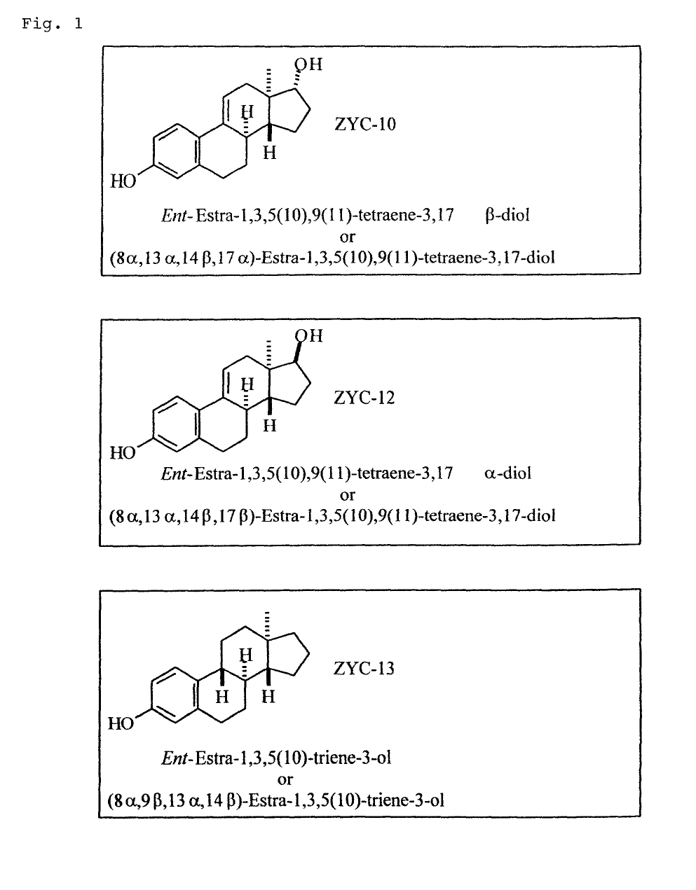

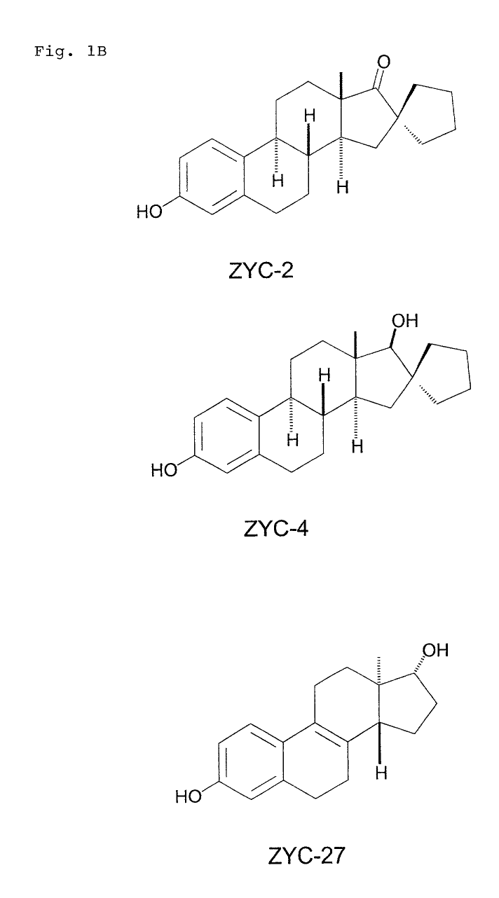

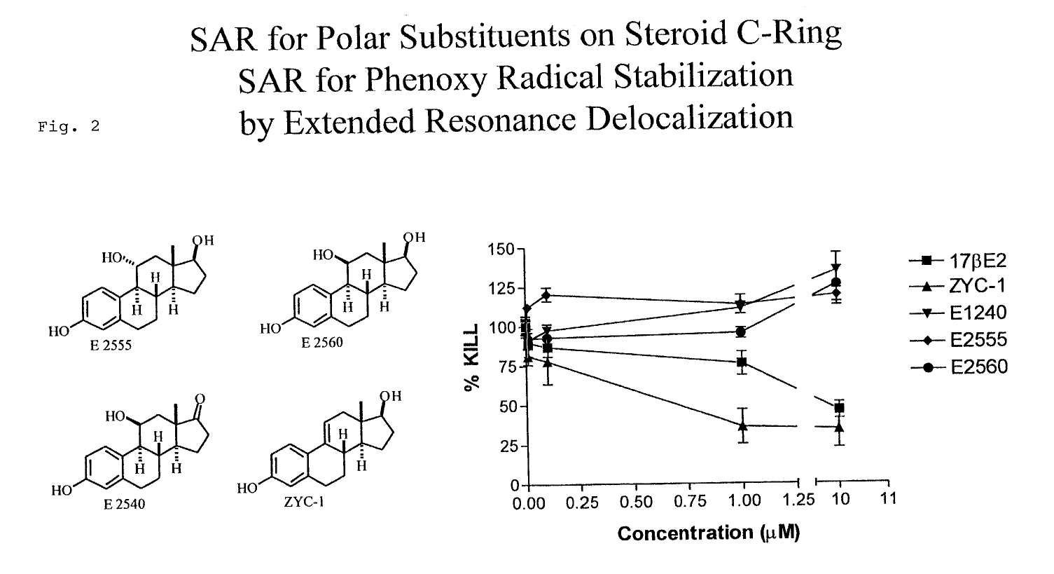

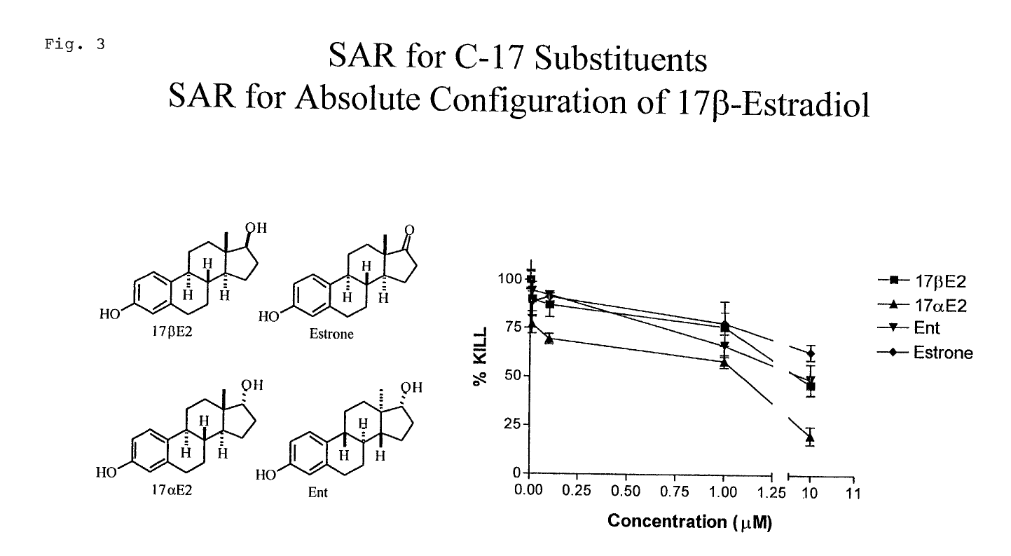

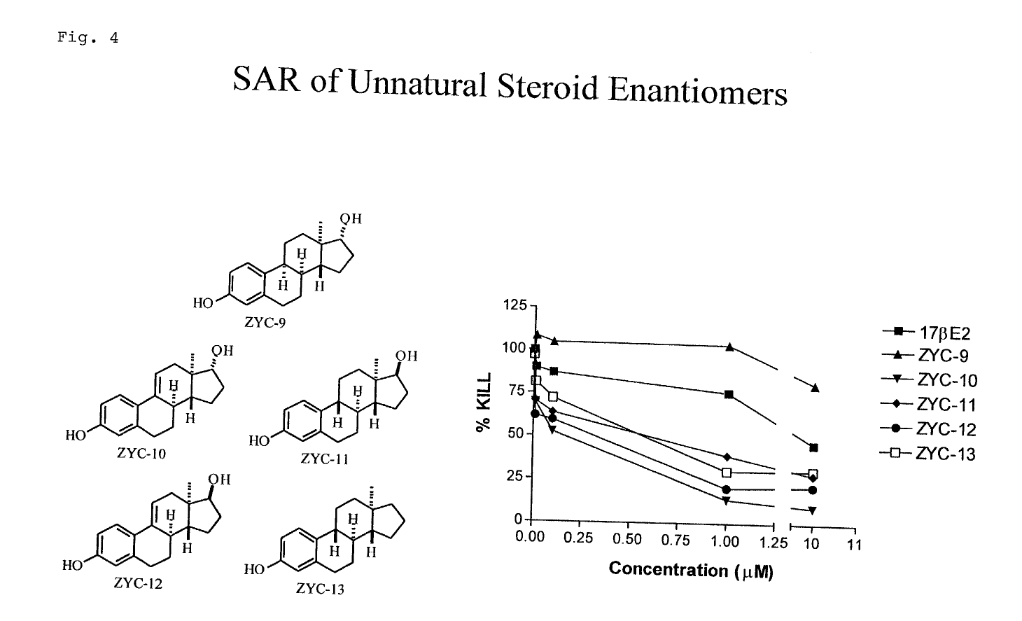

Referring now to FIG. 1 and the structures provided below, it is to be noted examples of some preferred enantiomeric compounds (denoted “Ent,” as further described herein), having unsaturated bonds in conjugation with the aromatic A-ring, include:

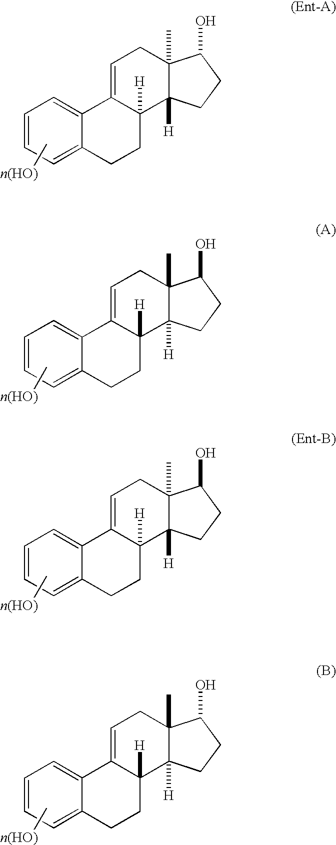

wherein n ranges from 1 to 4 (e.g., 1 or 2).

In this regard it is to be noted that, when present, the position of the hydroxy group (or more generally R^{z}) on the terminal D-ring, as well as the orientation thereof, may be other than herein described without departing from the scope of the present invention. For example, R^{z} (e.g., hydroxy group) may be in either the alpha or beta orientation when present.

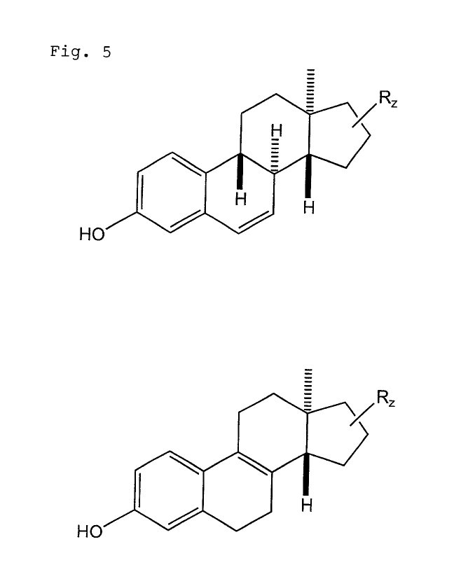

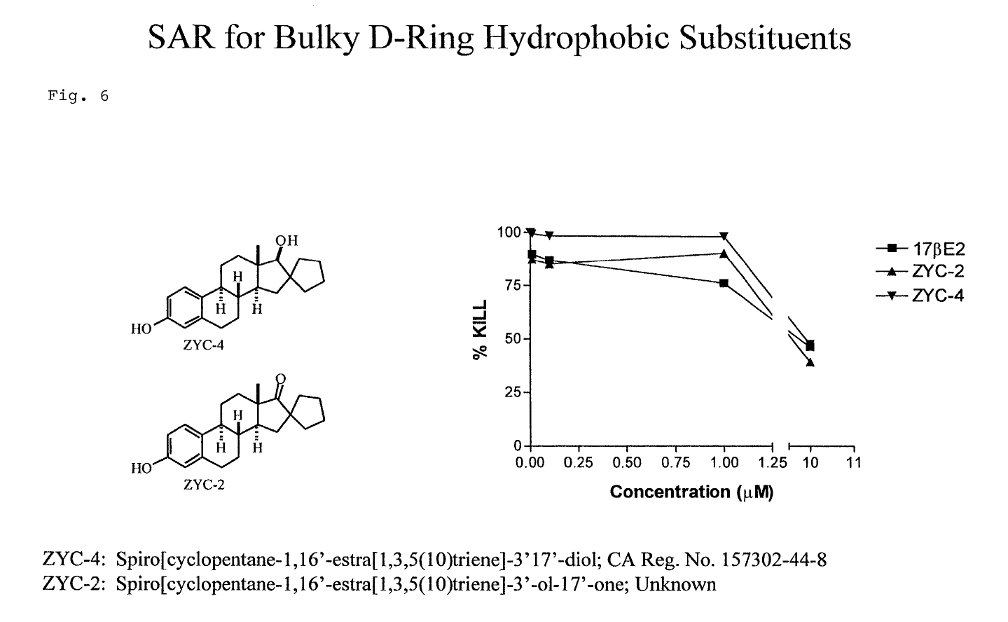

It is to be further noted that, when present, the position of unsaturated bond or bonds may be other than herein described without departing from the scope of the present invention. For example, referring now to FIG. 5 and the structures provided below, in other embodiments preferred compounds include:

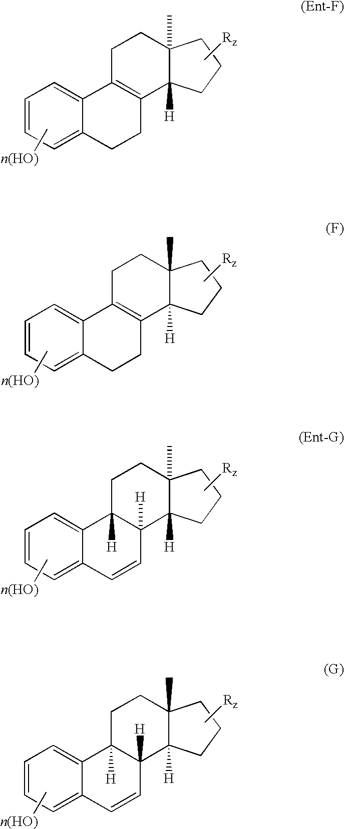

wherein n ranges from 1 to 4 (e.g., 1 or 2).

Additionally, it is to be noted that, in alternative embodiments, the ring adjacent the terminal aromatic ring may also be aromatic. Exemplary structures include:

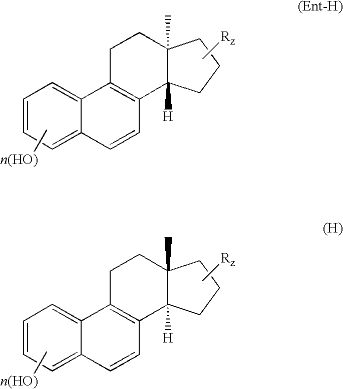

wherein n ranges from 1 to 4 (e.g., 1 or 2).

As previously noted, R^{z} generally represents one or more substituents, which are typically selected from hydrogen, hydroxy or oxo. However, R^{z} may additionally represent one or more other substituents selected from the group consisting of substituted or unsubstituted hydrocarbyl (e.g., hetero-substituted hydrocarbyl), halogens (e.g., fluoro, bromo, chloro), amides, sulfates, and nitrates, among other things. Alternatively, or additionally, Rz may represent an attached ring structure (e.g., cycloalkyl, cycloalkenyl, or heterosubstituted analogs thereof); that is, a ring structure attached by some linkage to the D-ring or directly thereto (e.g., a ring assembly, a fused ring, a spiro ring).

Furthermore, while in the structures provided herein the substituents at each chiral center are specifically indicated, these are not to be interpreted in a limiting sense. For example, one or more of the hydrogens may be replaced by a lower alkyl group (e.g., methyl, ethyl, propyl, etc.), while one or more methyl groups may in some instances be replaced by a substituent selected from the group consisting of substituted or unsubstituted hydrocarbyl, halogens (e.g., fluoro, bromo, chloro), amides, sulfates, and nitrates, among other things.

Compounds Lacking Extended Conjugation

As previously noted, in some embodiments, the present invention is directed to enantiomers of naturally-occurring steroids which do not have unsaturated bonds in conjugation with the aromatic A-ring. Such compounds include, for example:

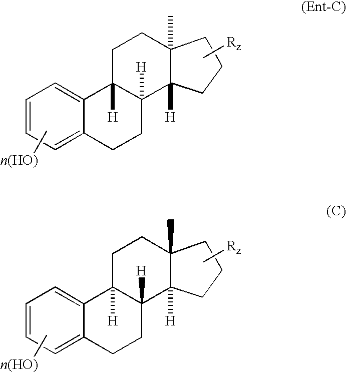

wherein n and R^{z} are as previously defined above.

Spiro-substituted Compounds

As previously noted, in some embodiments R^{z} may a substituent which forms an additional ring, which is bound or fused in some way to the D-ring of the estrogen compound. Accordingly, R^{z} may be a cycloalkyl (e.g., cyclopentyl) or a cycloalkenyl substituent, and more specifically in one preferred embodiment is a spiro substituent (e.g., cyclopentyl), wherein a carbon of the D-ring is also a carbon of the substituent (forming two bonds therein). Experience to-date suggests that, in this instance, both enantiomeric configurations have cytoprotective activity; that is, experience to-date suggests such a compound has cytoprotective activity in either the naturally-occurring estrogen configuration, or the enantiomeric configuration thereof (as well as the various diastereomeric configurations which are possible). Accordingly, the present invention is further directed to a compound, as well as the use thereof, having the formula (II):

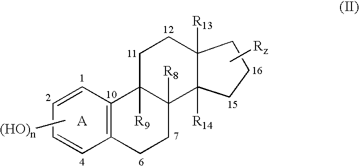

wherein

the compound optionally has one or more unsaturated bonds in conjugation with the aromatic A-ring between carbons 6 and 7, 8 and 9 or 9 and 11, in which event one or both of R⁸ and R⁹ will be absent;

n ranges from 1 to 4;

R⁸ and R⁹, when present, are independently hydrogen or alkyl;

R¹³ is hydrogen, substituted or unsubstituted hydrocarbyl, halo, amido, sulfate or nitrate;

R¹⁴ is hydrogen or alkyl;

R^{z} is substituted or unsubstituted cycloalkyl or cycloalkenyl, or substituted or unsubstituted heterocycloalkyl or heterocycloalkenyl.

Such compounds include, for example, those having the general structure (III):

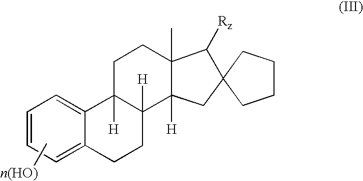

wherein n is as previously defined. Among some of the preferred embodiments are:

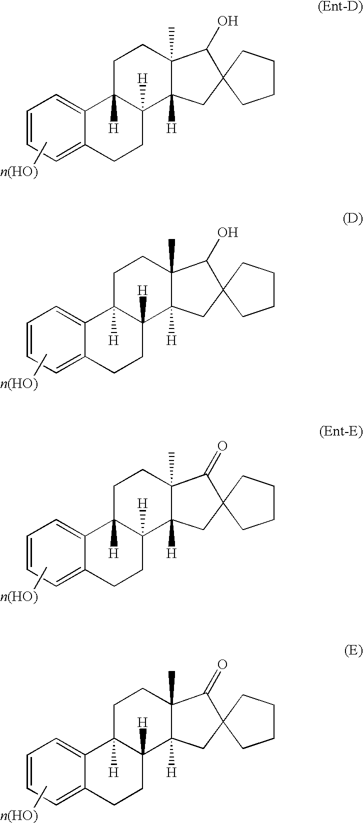

In this regard it is to be noted, however, that, in some embodiments of the above-described compound, when R^{z} is a spiro cyclopentyl substituent, C-3 is hydroxy substituted, and the configuration at C-8, C-9, C-13 and C-14 is that of the naturally-occurring enantiomer, C-17 is other than hydroxy-substituted, and in some cases R^{z} is also not oxo.

Additionally, it is to be further noted that in certain embodiments of the present process, wherein the above-described compound (i.e., that represented by formula II), when R^{z} is a spiro cyclopentyl substituent, C-3 is hydroxy substituted, and the configuration at C-8, C-9, C-13 and C-14 is that of the naturally-occurring enantiomer, R^{z}, if at C-17, is other than oxo.

Additional Substitution

Additionally, although not shown, it is to be understood that additional or alternative substitution may occur at any carbon in the structure without departing from the scope of the present invention. More specifically, it is to be understood that the present invention are generally directed to compounds, as well as the uses thereof, having the structures presented below:

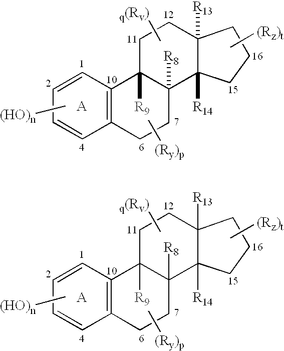

wherein R^{y} and R^{v} generally represent additional substituents on the B and C rings of the compound, and are generally as defined herein in the same manner as R⁸, R⁹, R¹⁴ or R^{z}, and further wherein p and q are 1 or 2.

Finally, it is to be noted that while typically a methyl group is present at the C,D-ring fusion, in some alternative embodiments this is not present (the methyl group being replace by, for example, hydrogen or some other substituent as described herein).

Administration/Application

Generally speaking, the process of the present invention involves the treatment of a population of cells in a subject (e.g., animal or human), in order to confer cytoprotection to that population, by the administration of an effective dose of the above-described compound. Experience to-date suggests such protection can be achieved at low plasma concentrations, concentrations which can be significantly lower than those needed for the non-substituted (i.e., non-R1 substituted) analogs of the present compounds. More specifically, a cytoprotective or even a neuroprotective effect can be achieved, in some cases, at plasma concentrations of less than about 10 μM, 1 μM, 500 nM, 100 nM, 10 nM, or even 1 nM (i.e., from about 0.1 nM to about 1 nM).

Administration of any of the compounds of the invention may be achieved by means standard in the art, and may include the use of a single compound or a mixture of cytoprotective compounds, their diastereomers (and in some cases their enantiomers as described herein), or pharmaceutically acceptable salts thereof. The recommended route of administration of the compounds of the present invention includes oral, intramuscular, transdermal, buccal, nasal, intravenous and subcutaneous. Methods of administering the compounds of the invention may be by dose or by controlled release vehicles.

Additionally, it is to be noted that, similar to the approach described by Simpkins et al. in U.S. Pat. No. 5,972,923 (incorporated herein by reference), a pharmaceutical preparation may also include, in addition to one or more compounds of the present invention, an additional antioxidant. As noted by Simpkins et al., in reference to compounds similar to those of the present invention, synergistic effects may be achieved in certain circumstances when such a combination is employed. For example, Simpkins et al. reports that estratrienes exhibit approximately a 1000-5000 fold enhancement in their cytoprotective effect when administered with the antioxidant, glutathione.

The present compounds are suitable, for example, in treating subjects suffering from trauma, chronic degenerative diseases or acute disease such as induced by an ischemic attack. Specific examples include Alzheimer's disease, Parkinson's disease, stroke, ischemia, heart attack or angioplasty, or brain or spinal cord trauma, hypoglycemia, anoxia, burns or surgeries that result in the loss of nutrient flow to the tissues. Other diseases that may be treatable with compounds of the current invention include: heart disease, including myocardial infarction, ophthalmologic diseases including macular degeneration, lens or retinal degeneration, formation of cataracts and glaucoma, alcoholism, alcohol withdrawal, drug-induced seizures vascular occlusion, epilepsy, cerebral vascular hemorrhage, hemorrhage; environmental excitotoxins, dementias of all type, drug-induced brain damage and other systemic or acute degenerative diseases characterized by necrotic or apoptotic cell death. To-date, there are no known cures and few therapies that slow the progression of these diseases. However, the present invention provides compounds which can be used as therapeutics or as prophylactics to treat, prevent or delay the onset of symptoms.

Certain embodiments of the present invention may further be applied to the procedure of tissue transplantation, prior, during or after removal or reperfusion of cells, tissues or organs or during storage of the cells, tissues or organs and is applicable to any of the cells in the body.

Preparation

Generally speaking, the compounds of the present invention may be prepared by means standard in the art. Specific details for the preparation of certain compounds, some of which are as heretofore unknown, are provided herein in the Examples, below.

Activity

The activity of the compounds of the present invention may be determined by means standard in the art (see, e.g., U.S. Pat. Nos. 5,972,923; 5,877,169; 5,859,001; 5,843,934; 5,824,672; and 5,554,601; see also P. S. Green et al., The Nonfeminizing Enantiomer of 17,β-Estradiol Exerts Protective Effects in neuronal Cultures and a Rat Model of Cerebral Ischemia, Endocrinilogy, 142(1), p. 400-06 (2001); all of which are incorporated herein by reference). Alternative methods for determining activity are described in detail herein in the Examples, below.

Definitions

As used herein, the following phrases or terms shall have the noted meanings; however, it is to be understood that these definitions are intended to supplement and illustrate, not preclude or replace, the definitions known to those of skill in the art.

“Hydroxy-substituted aromatic” or “hydyroxy-bearing aromatic” structure or ring, as well as variations thereof, refers to a terminal ring of a compound of the present invention which is both aromatic and substituted with one or more hydroxy groups. It is therefore to be understood that such phrases are intended to refer to compounds wherein the entire structure is aromatic (e.g., naphthalene, anthracene, and phenanthracene), as well as to compounds wherein only the terminal ring is aromatic (e.g., indan and 1,2,3,4-tetrahydronaphthlene) or where only one or two of the rings in a polycyclic structure are aromatic.

“Cytoprotection” refers to the protection of cells against cell death or cell damage that would otherwise occur in the absence of a protective agent, where the cell death or cell damage might be caused by any mechanical damage, nutritional deprivation (including oxygen deprivation), trauma, disease processes, damage due to exposure to chemicals or temperature extremes, aging or other causes.

“Neuroprotection” is one form of cytoprotection and refers to the inhibition of the progressive deterioration of neurons that lead to cell death.

“Enhanced” cytoprotective or neuroprotective activity refers to the increase in activity of the compounds of the present invention, as compared to the naturally occurring analogs thereof or alternative to analogs wherein additional conjugation with the terminal aromatic ring is not present.

An “estrogen compound” refers to any of the structures described in the 11th Edition of “Steroids” from Steraloids Inc., Wilton N.H., incorporated herein by reference. Included in this definition are isomers and enantiomers, including non-steroidal estrogens formed by modification or substitution of the compounds in the Steraloid reference. Other estrogen compounds included in this definition are estrogen derivatives, estrogen metabolites and estrogen precursors, as well as those molecules capable of binding cell-associated estrogen receptors as well as other molecules where the result of binding specifically triggers a characterized estrogen effect. Also included are mixtures of more than one estrogen, where examples of such mixtures are provided in, for example, U.S. Pat. No. 5,972,923. Examples of α-estrogen structures having utility either alone or in combination with other agents are provided in, for example, U.S. Pat. No. 5,972,923 as well.

A “non-estrogen compound” refers to a compound other than an estrogen compound as defined above.

The terms “17-E2,” “β-estradiol,” “17β-estradiol,” “β-17-E2,” “17β-E2,” “E2,” “17βE2,” and “βE2,” are intended to be synonymous. Similarly, the terms “α17-E2,” “α-17-E2,” “α-estradiol,” “17α-estradiol,” “17αE2,” and “αE2,” as defined here and in the claims, are intended to be synonymous and correspond to the α-isomer of 17β-estradiol.

“E-3-ol” refers to estra-1,3,5(10)-trien-3-ol.

The terms “polycyclic phenolic compound,” “polycyclic compounds” or “polycyclic phenols” as used herein are generally synonymous and are defined, for example, in U.S. Pat. No. 5,859,001 (herein incorporated by reference); the terms generally include any compound having a phenolic A-ring and may contain 2, 3, 4 or even more additional ring structures exemplified by the compounds described herein.

A “steroid” refers to a compound having numbered carbon atoms arranged in a 4-ring structure (see, e.g., J. American Chemical Society, 82:5525-5581 (1960); and, Pure and Applied Chemistry, 31:285-322 (1972)).

A “cytodegenerative” disorder or disease refers to a disorder or disease related to cell death or cell damage, which might be caused by any mechanical damage, nutritional deprivation (including oxygen deprivation), trauma, disease processes, damage due to exposure to chemicals or temperature extremes, aging or other causes.

A “neurodegenerative disorder” or “disease” refers to a disorder or disease in which progressive loss of neurons occurs either in the peripheral nervous system or in the central nervous system. Examples of neurodegenerative disorders include: chronic neurodegenerative diseases, such as Alzheimer's disease, Parkinson's disease, Huntington's chorea, diabetic peripheral neuropathy, multiple sclerosis, amyotrophic lateral sclerosis; aging; and acute neurodegenerative disorders including: stroke, traumatic brain injury, schizophrenia, peripheral nerve damage, hypoglycemia, spinal cord injury, epilepsy, and anoxia and hypoxia.

“Linker” embraces a saturated or partially unsaturated moiety, typically a hydrocarbylene (e.g., alkylene, akenylene, akynylene), or alternatively a substituted hydrocarbylene (e.e., wherein a carbon in the main chain has been substituted by a heteroatom, such as oxygen or sulfur), interposed between the core ring structure X and the modifying hydrophobic substituent, R¹, or alternatively between the core ring structure X and another substituent (e.g., R², R³, etc.).

“Hydrocarbyl” embrace moieties consisting exclusively of the elements carbon and hydrogen, in a straight or branched chain, or alternatively a cyclic structure, which may optionally be substituted with other hydrocarbon, halo (e.g., chlorine, fluorine, bromine) or hetero (e.g., oxygen, sulfur) substituents. These moieties include alkyl, alkenyl, alkynyl and aryl moieties, as well as alkyl, alkenyl, alkynyl and aryl moieties substituted with other aliphatic or cyclic hydrocarbon groups such as, for example, alkaryl, alkenaryl and alkynaryl.

The alkyl groups described herein are, in some embodiments, preferably lower alkyl containing from about 1 to about 6 carbon atoms in the principal chain. They may be straight or branched chains and include methyl, ethyl, propyl, isopropyl, butyl, isobutyl, tertiary butyl, pentyl, hexyl and the like. They may be substituted with aliphatic or cyclic hydrocarbon moieties or hetero-substituted with the various substituents defined herein.

The alkenyl groups described herein are, in some embodiments, preferably lower alkenyl containing from about 2 to about 6 carbon atoms in the principal chain. They may be straight or branched chains and include ethenyl, propenyl, isopropenyl, butenyl, isobutenyl, pentenyl, hexenyl, and the like. They may be substituted with aliphatic or cyclic hydrocarbon moieties or hetero-substituted with the various substituents defined herein.

The alkynyl groups described herein are, in some embodiments, preferably lower alkynyl containing from about 2 to about 6 carbon atoms in the principal chain. They may be straight or branched chain and include ethynyl, propynyl, butynyl, isobutynyl, pentynyl, hexynyl, and the like. They may be substituted with aliphatic or cyclic hydrocarbon moieties or hetero-substituted with the various substituents defined herein.

The term “cycloalkyl” is used herein to refer to a saturated cyclic non-aromatic hydrocarbon moiety having a single ring or multiple condensed rings. Exemplary cycloalkyl moieties include, for example, cyclopentyl, cyclohexyl, cyclooctanyl, etc.

The term “cycloalkenyl” is used herein to refer to a partially unsaturated (i.e., having at least one carbon-carbon double bond), cyclic non-aromatic hydrocarbon moiety having a single ring or multiple condensed rings. Exemplary cycloalkenyl moieties include, for example, cyclopentenyl, cyclohexenyl, cyclooctenyl, etc.

“Substituted cycloalkyl” and “substituted cycloalkenyl” refer to cycloalkyl and cycloalkenyl moieties, respectively, as just described wherein one or more hydrogen atoms to any carbon of these moieties is replaced by another group such as a halogen, alkyl, alkenyl, alkynyl, substituted alkyl, substituted alkenyl, substituted alkynyl, aryl, substituted aryl, cycloalkyl, cycloalkenyl, substituted cycloalkyl, substituted cycloalkenyl, heterocyclo, substituted heterocyclo, heteroaryl, substituted heteroaryl, alkoxy, aryloxy, boryl, phosphino, amino, silyl, thio, seleno and combinations thereof.

“Terminal,” as used in the context of the hydroxy-substituted aromatic ring structure, generally refers to the position of the ring relative to the rest of the molecule, the ring being located at or proximate one end of the molecule, such as in the case of the tetracyclic estrogen compounds (the hydroxy-substituted aromatic ring being the A-ring of the compound).

The following Examples set forth one approach for preparing and testing compounds in accordance with the present invention. These Examples are intended to be illustrative of compounds preferred for certain embodiments only, as well as their respective activity in the protection of neuron. Generally speaking, however, it is understood that in many cases drugs which protect neurons are also active in protecting non-neuronal cells. Accordingly, in advancing the understanding of the structural requirements for compositions capable of inducing neuroprotection, these results in turn provide the basis for designing novel drugs that have enhanced cytoprotective properties, as well. Therefore, these Examples should not be viewed in a limiting sense.

### EXAMPLE 1

### Preparation of Ent-estra-1,3,5(10),9(11)-tetraene-3,17β-diol

The preparation of the above-referenced compound is illustrated by the following reaction scheme. Details for the reactions carried out in each of the indicated steps, in order to prepare the various intermediate compounds and ultimately the above-referenced compound, are provided below.

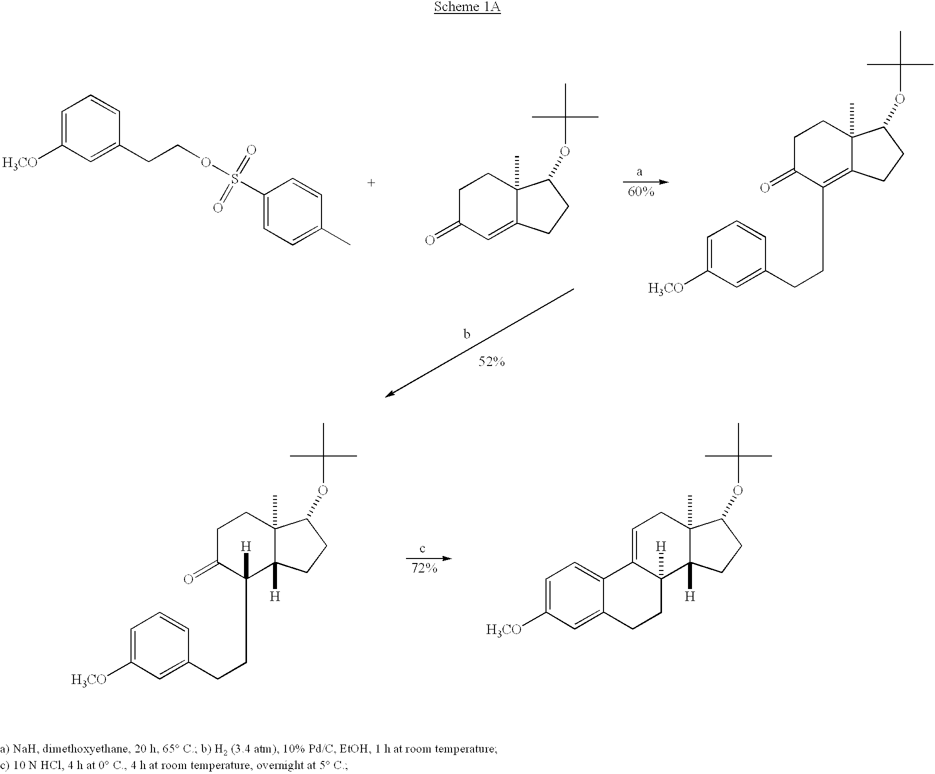

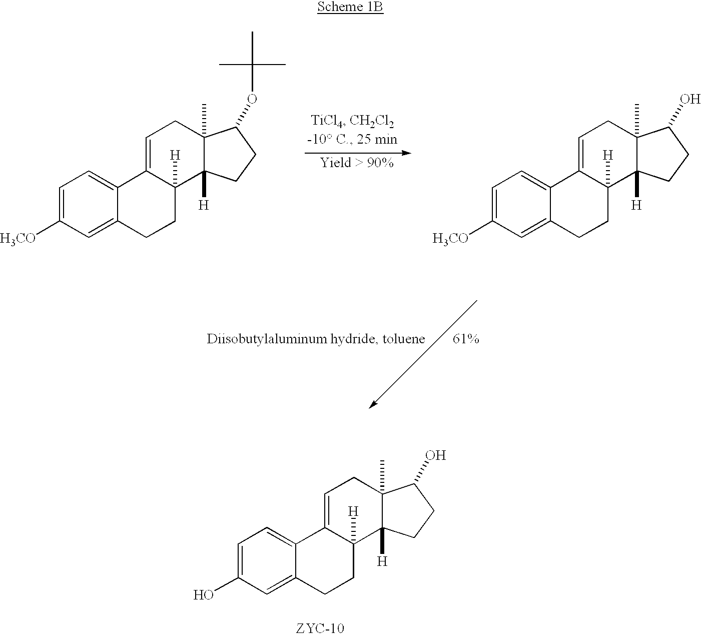

### (1R-cis)-1-(1,1-Dimethylethoxy)-1,2,3,6,7,7a-hexahydro-4-[2-(3-methoxyphenyl)ethyl]-7a-methyl-5H-inden-5-one

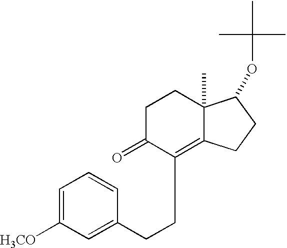

To a suspension of 1.21 g of 60% NaH (30.25 mmole, washed with anhydrous hexanes (2×10 mL) in 48 mL anhydrous dimethoxyethane, was added (1R-cis)-1-(1,1-dimethylethoxy)-1,2,3,6,7,7a-hexahydro-7a-methyl-5H-inden-5-one (4.5 g, 20.27 mmol, Chemical Abstracts Registry Number [61217-34-3]) under N₂. The mixture was heated and stirred at 65° C. under N₂ for 20 h and became dark brown. 3-Methoxybenzeneethanol 4-methylbenzenesulfonate (7.09 g, 23.17 mmol, Chemical Abstracts Registry Number [25112-95-2]) dissolved in 40 mL of dimethoxyethane was then added quickly (15 min) into the above dark brown reaction. Heating at 65° C. under N₂ was continued for 20 h. After cooling the reaction flask with ice, 50 mL of saturated NaH₂PO₄ solution was added and the red orange solution was extracted with methylene chloride. The combined extracts were washed with saturated brine, dried with Na₂SO₄, and the solvent was removed to yield 9.01 g of deep orange crude product. Purification by chromatography (silica gel eluted with 3%, 4%, 5%, 6%, 7.5% ethyl acetate in hexanes) gave pure product (4.33 g, 60% yield) that had: [α]_{D}−41.76 (c=0.455, CHCl₃). UV (EtOH), λₘₐₓ 251 nm, ε=15,000. IR(film) 1661, 1652, 1608, 1557, 2834 cm⁻¹. ¹H NMR(CDCl₃) δ1.06 (s, 3H, CH₃), 1.20 (s, 9H, C(CH₃)₃), 3.48-3.43 (q, J=10.2 Hz, 7.5 Hz, 1H, CHOC(CH₃)₃), 3.83 (s, 3H, OCH3), 6.72-6.75 (m, 1H, Ar—H), 6.76-6.80 (m, 2H, Ar—H), 7.21 (t, 1H, J=7.8 Hz, 1H, Ar—H). ¹³C NMR(CDCl₃) δ198.69, 7.21 (t, 1H, J=7.8 Hz, 1H, Ar—H). ¹³C NMR(CDCl₃) δ198.69, 169.65, 159.42, 143.74, 131.51, 129.02, 121.34, 114.73, 110.84, 79.66, 72.77, 54.98, 44.52, 34.45, 33.93, 33.46, 29.55, 28.45, 27.51, 25.13, 15.54. MS (C₂₃H₃₂O₃): m/z 356(M⁺), 300, 222, 179, 166, 148, 135, 122, 107, 91, 57.

### [1R-(1α,3aβ,4α,7aα)]-1-(1,1-Dimethylethoxy)octahydro-4-[2-(3-methoxyphenyl)ethyl]-7a-methyl-5H-inden-5-one

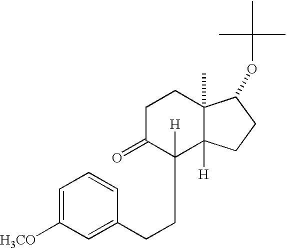

To a solution of (1R-cis)-1-(1,1-dimethylethoxy)-1,2,3,6,7,7a-hexahydro-4-[2-(3-methoxyphenyl)ethyl]-7a-methyl-5H-inden-5-one (3.76 g, 10.56 mmol) in 360 mL of ethanol was added 0.96 g of 10% Pd/C. Hydrogenation was carried out under 3.4 atm H₂ for 1 h. The catalyst was removed by filtration with washing by ethanol. After removing the solvent, the product was purified by chromatography (silica gel eluted with 2.5%, 3%, 3.5%, 7.5% EtOAc in hexanes). The product (1.96 g, 52% yield) eluted in 3.5% EtOAc in hexanes and had: [α]²⁰_{D}−24.7 c=0.215, EtOH). UV (EtOH) λₘₐₓ 280 nm, ε=1,800; λₘₐₓ 273 nm, ε=1,900. ¹H NMR (CDCl₃) δ1.01 (s, 3H, CH₃), 1.13 (s, 9H, C(CH₃)₃), 3.38 (t, J=8.4 Hz, CHOC(CH₃)₃), 3.79 (s, 3H, OCH3), 6.72-6.78 (m, 3H, Ar—H), 7.17-7.22 (m, 1H, Ar—H). ¹³C NMR (CDCl₃) δ215.37, 159.68, 143.41, 129.34, 120.90, 114.32, 111.27, 79.60, 78.23, 72.45, 55.03, 52.86, 49.41, 47.71, 47.56, 41.47, 37.69, 36.06, 34.98, 33.32, 32.96, 32.34, 31.28, 29.30, 29.20, 28.53, 28.42, 24.58, 21.67, 20.59, 12.53. MS (C₂₃H₃₄O₃): m/z 358(M⁺) , 302, 224, 181, 167, 147, 134, 122, 107, 93, 57.

### [1R-(1α,3aβ,4β,7aα)]-1-(1,1-Dimethylethoxy)octahydro-4-[2-(3-methoxyphenyl)ethyl]-7a-methyl-5H-inden-5-one

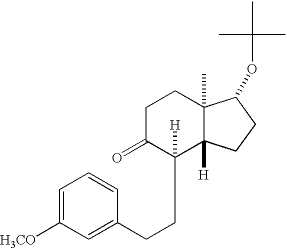

The diastereomeric 4β-epimer (100 mg) was also obtained from the chromatography. ¹H NMR(CDCl₃) δ1.02 (s, 3H, CH₃), 1.14 (s, 9H, C(CH₃)₃), 3.45 (t, J=8.7 Hz, CHO(CH₃)₃), 3.80 (s, 3H, OCH₃), 6.72-6.81 (m, 3H, Ar—H), 7.19 (t, J=7.8 Hz, 1H, Ar—H). ¹³C NMR(CDCl₃) δ213.07, 159.74, 144.59, 129.31, 120.89, 114.08, 111.16, 79.40, 72.51, 55.09, 49.99, 49.65, 42.75, 38.01, 35.91, 33.46, 31.70, 28.57, 28.39, 24.48, 11.03. MS (C₂₃H₃₄O₃): m/z 358(M⁺), 301, 245, 224, 181, 167, 134, 121, 93, 57.

### Ent-(17β)-17-(1,1-dimethylethoxy)-3-methoxyestra-1,3,5(10),9(11)-tetraene

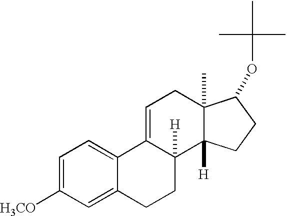

[1R-(1α,3aβ,4α,7aα]-1-(1,1-Dimethylethoxy)octahydro-4-[2-(3-methoxyphenyl)ethyl]-7a-methyl-5H-inden-5-one (1.21 g) was dissolved in 30 mL of methanol, cooled to 0° C. for 20 min. with and ice/salt bath and 3.2 mL of 10 N HCl was added quickly. Stirring was continued at 0° C. for 4 h, then at room temperature for an additional 4 h during which time a white precipitate formed. The reaction mixture was stirred in a cold room (5° C.) overnight. The crude product (0.91 g, m.p. 124-126° C.) was obtained by filtration and recrystallized from methanol and methylene chloride to yield pure product (0.83 g, 72%) that had: m.p. 128-129° C.; [α]_{D}−109.9 c=0.356, CHCl₃). UV λₘₐₓ (EtOH) 263 nm, ε=14,900. IR(KBr) 1626, 1604, 1568 1255, 1197, 1116 cm⁻¹. ¹H NMR(CDCl₃) δ0.78 (s, 3H, CH₃), 1.17 (s, 9H, C(CH₃)₃), 3.54 (t, J=8.7 Hz, 1H, CHOC(CH₃)₃), 3.79 (s, 3H, OCH₃), 6.12 (d, J=5.4 Hz, C═C—H), 6.59 (d, J=2.7 Hz, Ar—H), 6.71 (dd, J=8.7 Hz, 2.7 Hz, 1H, Ar—H), 7.53 (d, J=8.7 Hz, 1H, Ar—H). ¹³C NMR(CDCl₃) δ158.34, 137.60, 135.06, 127.72, 125.17, 118.04, 113.29, 112.65, 82.60, 80.81, 72.24, 55.18, 47.30, 41.08, 40.96, 39.49, 38.89, 31.17, 30.11, 28.70, 28.15, 24.76, 24.29, 11.55. Anal. Calc'd. for C₂₃H₃₂O₂: C, 81.13; H, 9.47. Found: C, 81.26; H, 9.47.

### Ent-(17β)-17-(1,1-dimethylethoxy)-3-methoxyestra-1,3,5(10),8-tetraene

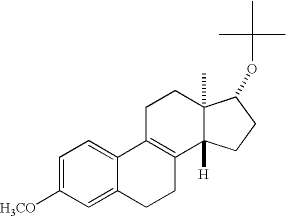

This compound was isolated as a minor product. ¹H NMR (CDCl₃) δ0.93 (s, 3H, CH₃), 1.18 (s, 9H, C(CH₃)₃), 2.71 (m, 1H, CH), 3.57 (t, J=6.3 Hz, 1H, CHOC(CH₃)₃), 3.80 (s, 3H, OCH₃), 6.68-6.74 (m, 2H, Ar—H), 7.13 (d, J=8.4 Hz, 1H, Ar—H).

### Ent-(17β)-3-methoxyestra-1,3,5(10),9(11)-tetraen-17-ol

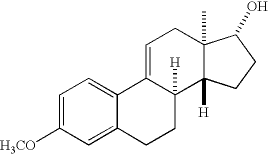

To the stirred solution of ent-(17β)-17-(1,1-dimethylethoxy)-3-methoxyestra-1,3,5(10),9(11)-tetraene (100 mg, 0.294 mmol) in 4 mL of anhydrous methylene chloride at −10° C., was quickly added 0.375 mL of 1M TiCl₄ in methylene chloride. After 15 min, 4 mL of water was added to stop the reaction. The reaction mixture was extracted with methylene chloride and the combined extracts were washed with brine and dried over anhydrous sodium sulfate. After removal of the solvent, 90 mg of the product was obtained. This material can be used directly for removal of the methoxy group or it can be purified by chromatography (silica gel eluted 15% ethyl acetate in hexanes).

¹H NMR(CDCl₃) δ0.80 (s, 3H, CH₃), 2.84 (m, 2H, CH₂), 3.79 (s, 3H, OCH₃), 3.82 (m, 1H, CHOH), 6.13 (t, 1H, J=2.1 Hz, C═C—H), 6.60 (d, 1H, J=2.7 Hz, Ar—H), 6.72 (dd, 1H, J=2.7 Hz, 8.7 Hz, Ar—H), 7.54 (d, 1H, J=8.7, Ar—H). ¹³C NMR(CDCl₃) δ10.85, 23.87, 28.15, 30.02, 30.71, 38.80, 38.91, 41.50, 47.35, 55.18, 82.02, 112.69, 113.35, 117.54, 125.23, 127.57, 135.12, 137.58, 158.47.

### Ent-estra-1,3,5(10),9(11)-tetraene-3,17,β-diol

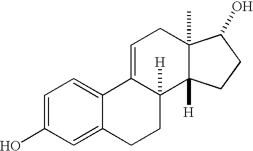

Under nitrogen, to a solution of ent-(17β)-3-methoxy-1,3,5(10),9(11)-tetraen-17-ol (120 mg, 0.423 mmol) in 4 mL anhydrous toluene, was added 3 mL of 1.5 M DIBAL in toluene. The reaction was refluxed overnight and after cooling to room temperature, ice was added. The reaction mixture was acidified with 3 N HCl and extracted with ethyl acetate. The combined extracts were washed with brine and dried over anhydrous sodium sulfate. After removal of the solvent, the crude product was purified by chromatography (silica gel eluted with 35% ethyl acetate in hexanes) and the product was crystallized from methylene chloride-hexanes to yield a white solid (70 mg, 61% yield) that had: m.p. 191-192° C., [α]²⁵_{D}−138.4 c=0.305, dioxane).

UV (EtOH) λₘₐₓ 265 nm, ε=11,900. ¹H NMR(CDC13:CD3COCD3; 1:1) δ0.81 (s, 3H, CH3), 2.77-2.82 (m, 2H, CH2), 3.35 (d, 1H, J=5.1 Hz, CH) 3.80 (m, 1H, CHOH), 6.08 (d,1H, J=5.1 Hz, C═C—H), 6.56 (d, 1H, J=2.7 Hz, Ar—H), 6.65 (dd,1H, J=8.7 Hz, 2.7 Hz, Ar—H), 7.45 (d, J=8.7 Hz, Ar—H). ¹³C NMR (CDC13:CD3COCD3; 1:1) δ10.18, 23.20, 27.59, 28.29, 29.83, 38.30, 38.39, 40.87, 46.79, 80.82, 113.16, 114.44, 116.25, 124.55, 125.97, 134.67, 130.92, 155.28. MS (Cl8H22O2): m/z 270 (M+), 211, 181, 169, 157, 149, 129, 111, 97, 83, 69.

### EXAMPLE 2

### Preparation of Ent-estra-1,3,5(10),8-tetraene-3,17β-diol

The preparation of the above-referenced compound is illustrated by the following reaction scheme. Details for the reactions carried out in each of the indicated steps, in order to prepare the various intermediate compounds and ultimately the above-referenced compound, are provided below.

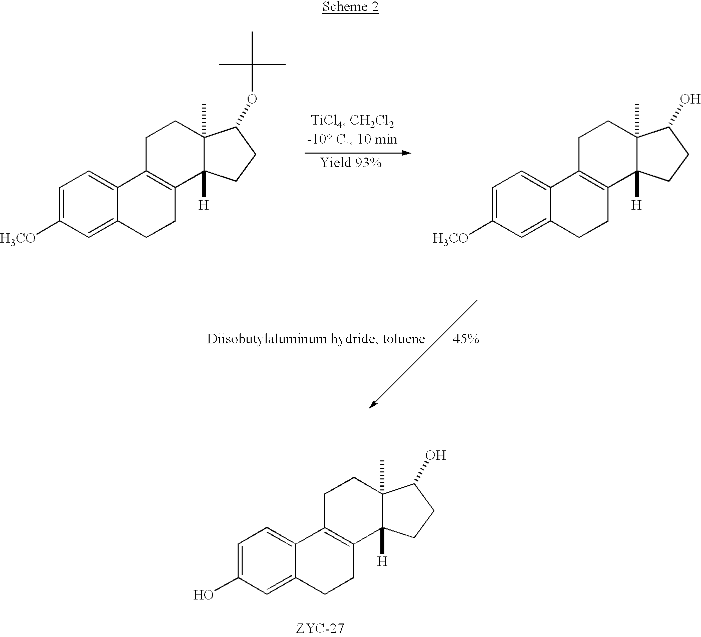

### Ent- (17β)-3-methoxyestra-1,3,5 (10) 8-tetraen-17-ol

To ent-(17β)-17-(1,1-dimethylethoxy)-3-methoxyestra-1,3,5(10),8-tetraene (90 mg, 0.265 mmol) in 3 mL of anhydrous methylene chloride cooled to −5° C. to −10° C. for 10 mins., was added 0.3 mL of titanium tetrachloride (1M in methylene chloride) over 1-2 min. The reaction mixture became orange. After 5 min, 1 mL of water was added and the color disappeared. The reaction was extracted with methylene chloride and the combined extracts were washed with brine and dried over anhydrous sodium sulfate. After removal of the solvent, the product (70 mg, 93% yield) was obtained.

¹H NMR(CDCl₃) δ1.00 (s, 3H, CH₃), 2.73 (t, J=8.4 Hz, 2H, CH₂), 3.80 (s, 3H, OCH₃), 3.84 (t, J=6 Hz, CHOH), 6.70 (s, 1H, Ar—H), 6.73 (d, J=8.1 Hz, 1H, Ar—H), 7.13 (d, J=8.1 Hz, 1H, Ar—H).

Ent-estra-1,3,5(10),8-tetraene-3,17β-diol

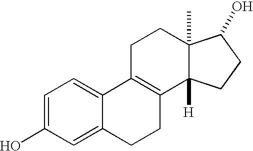

Ent-(17β)-3-methoxyestra-1,3,5(10),8-tetraen-17-ol (70 mg, 0.246 mmol) dissolved in anhydrous toluene (7 mL) was added to diisobutylaluminum hydride (2.25 mmol, 1.5 mL of a 1.5 M solution in toluene) under nitrogen. The reaction was refluxed overnight, cooled to room temperature and ice was added. Stirring was continued as the reaction warmed to room temperature and a solid formed. The reaction was acidified with 2.5N HCl and extracted with ethyl acetate. The combined extracts were washed with brine and dried over anhydrous sodium sulfate. After removal of solvent, a pale yellow oil was obtained which was purified twice by chromatography (silica gel eluted with 30% ethyl acetate in hexanes) to give a solid (30 mg, 45%) that had: m.p.86-96° C. [α]_{D}−99.45 c=0.365, CHCl₃).

UV _{λmax} 273 nm, ε=16,200. ¹H NMR(CHCl₃) δ1.00 (s, 3H, CH₃), 2.68 (t, J=8.1 Hz, 2 Hz, CH₂), 3.86-3.88 (q, J=5.1 Hz, CHOH), 5.83 (OH), 6.61-6.67 (m, 2H, Ar—H), 7.04-7.07 (d, J=8.1 Hz, Ar—H). ¹³C NMR (CHCl₃) 153.87, 137.27, 134.28, 129.30, 123.82, 122.97, 114.52, 112.59, 80.86, 48.09, 43.56, 32.17, 29.63, 29.16, 28.72, 28.24, 22.24, 18.49. MS (C₁₈H₂₂O₂): m/z 270(M⁺), 237, 211, 157, 81, 69.

### EXAMPLE 3

### Preparation of Ent-estra-1,3,5(10),9(11)-tetraene-3,17β-diol

The preparation of the above-referenced compound is illustrated by the following reaction scheme. Details for the reactions carried out in each of the indicated steps, in order to prepare the various intermediate compounds and ultimately the above-referenced compound, are provided below.

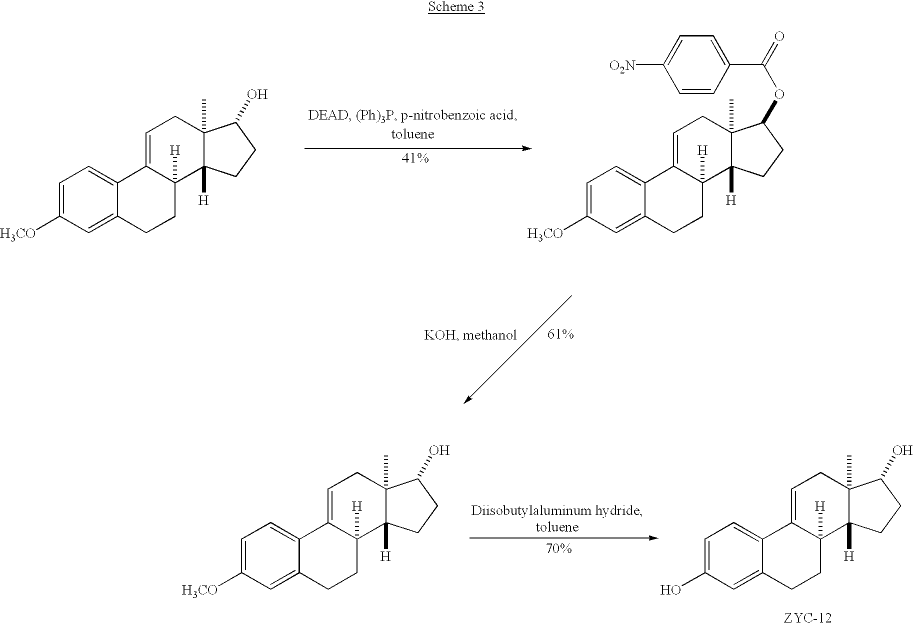

### Ent-(17α)-3-methoxyestra-1,3,5(10),9(11)-tetraen-17-ol 4-nitrohenzoate

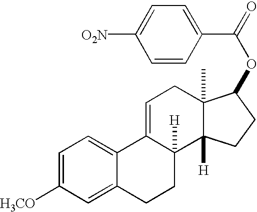

A mixture of ent-(17β)-3-methoxyestra-1,3,5(10),9(11)-10 tetraen-17-ol (0.24 g, 0.845 mmol), 4-nitrobenzoic acid (0.305 g, 1.84 mmol), triphenylphosphine (0.49 g, 1.87 mmol) and diethyl azodicarboxylate (0.45 g, 2.58 mmol) in 7 mL of anhydrous toluene was heated at 80° C. for 6 h. After the removal of the solvent, the residue was purified by chromatography (silica gel eluted with 10% ethyl acetate in hexanes) to obtain the product (150 mg, 41% yield).

¹H NMR(CHCl₃) δ0.90(s, 3H, CH₃), 2.87-2.89 (m, 2H, CH₂), 3.80 (s, 3H, OCH₃), 6.15 (t, 1H, C═C—H), 6.62 (d, 1H, J=2.7 Hz, Ar—H), 6.72 (dd, 1H, J=8.7 Hz, 2.7 Hz, ArH) , 7.55 (d, 1H, J=8.7, Ar—H), 8.18-8.31 (m, 4H, Ar—H).

### Ent-(17α)-3-methoxyestra-1,3,5(10),9(11)-tetraen-17-ol

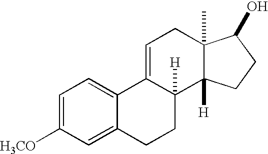

Ent-(17β)-3-methoxy-1,3,5(10),9(11)-tetraen-17-ol 4-nitrobenzoate (150 mg, 0.346 mmol) in 6 mL of 3% methanolic potassium hydroxide and 4 mL of THF was stirred at room temperature for 1 h. Then the reaction was acidified with 3N HCl (1.5 mL) and the solvent was removed. The residue was purified by chromatography (silica gel eluted with 20% ethyl acetate in hexanes) to give the product (60 mg, 61% yield).

¹H NMR (CDCl₃) δ0.71 (s, 3H, CH₃), 2.82-2.84 (m, 2H, CH₂), 3.78 (s, 3H, OCH₃), 3.84-3.86 )d, 1H, J=5.1 Hz, CHOH), 6.17 (t, 1H, J=2.7 Hz, C═C—H), 6.60 (d, 1H, J=2.4 Hz, Ar—H), 6.71 (dd, 1H, J=8.7 Hz, 2.4 Hz, Ar—H), 7.54 (d, 1H, J=8.7 Hz, Ar—H). ¹³C NMR (CHCl₃) δ17.37, 24.92, 29.09, 30.09, 32.66, 33.08, 38.98, 43.89, 45.43, 55.13, 79.49, 112.63, 113.27, 117.90, 125.15, 127.66, 134.79, 137.55, 158.31.

### Ent-estra-1,3,5(10),9(11)-tetraene-3,17α-diol

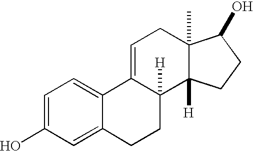

Under nitrogen, to 1.5 mL of 1.5 M DIBAL (2.25 mmol) in toluene was added 60 mg (0.21 mmol) of ent-(17α)-3-methoxyestra-1,3,5(10),9(11)-tetraen-17-ol in 3 mL of anhydrous toluene. The reaction was refluxed and stirred overnight. After cooling to room temperature, ice was added, the reaction mixture was acidified with 3 N HCl (3 mL) and then it was extracted with ethyl acetate. The combined extracts were washed with brine and dried over anhydrous sodium sulfate. After removal of the solvent, the crude residue was purified by chromatography (silica gel) and the product was crystallized from acetone-hexane to obtain pure product (40 mg , 70% yield) that had: m.p. 239-241° C.; [α]_{D}−131.3 c=0.265, dioxane).

UV (EtOH) λₘₐₓ 263 nm, ε=15,100. ¹H NMR (CHCl₃:CD₃COCD₃; 2:1) δ0.72 (s, 3H, CH₃), 2.73-2.78 (m, 2H, CH₂), 3.85 (t, 1H, J=5.4 Hz, CHOH), 6.14 (t, 1H, J=2.4 Hz, C═C—H), 6.56 (d, 1H, J=3.0 Hz, Ar—H), 6.65 (dd, 1H, J=8.7 Hz, 3.0 Hz, Ar—H), 7.47 (d, 1H, J=8.7 Hz, Ar—H). ¹³C NMR (CHCl₃;CD₃COCD₃; 2:1) δ16.77, 24.42, 29.91, 31.93, 32.58, 38.59, 43.31, 44.87, 78.40, 113.20, 114.50, 116.91, 124.58, 126.23, 134.32, 136.99, 155.202. Anal.Calc'd. for C₁₈H₂₂O₂: C, 79.96: H, 8.20. Found: C, 79.77: H, 8.37.

### EXAMPLE 4

### Preparation of Ent-estra-1,3,5(10)-trien-3-ol

The preparation of the above-referenced compound is illustrated by the following reaction scheme. Details for the reactions carried out in each of the indicated steps, in order to prepare the various intermediate compounds and ultimately the above-referenced compound, are provided below.

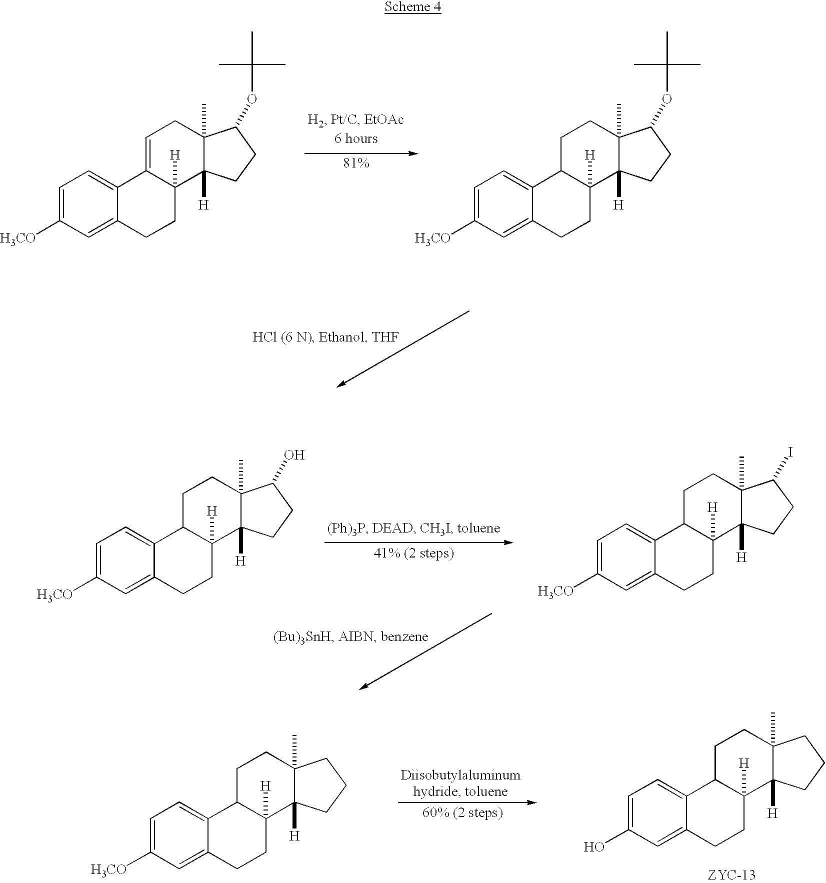

### Ent-(17β)-17-(1,1-dimethylethoxy)-3-methoxyestra-1,3,5(10)-triene

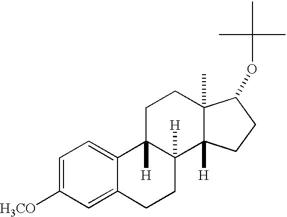

To a solution of ent-(17β)-17-(1,1-dimethylethoxy)-3-methoxyestra-1,3,5(10)-triene (1.08 g) in 74 mL of EtOAc was added 200 mg of 10% Pd/C. Hydrogenation was carried out under 3.4 atm of H₂ for 6 h. After removing the catalyst and solvent, the crude product (1.27 g) was purified by chromatography (silica gel eluated with 1% diethyl ether in hexanes to obtain product (0.88 g, 81% yield) that had: [α]_{D}−61.86 (c=0.485, CHCl₃).

UV (EtOH) λₘₐₓ 273 nm, ε=2,300; 277 nm, ε=2,300; 287 nm, ε=2,050. IR(KBr) 1611, 1575, 1198 cm⁻¹. ¹H NMR(CHCl₃) δ0.75 (s, 3H, CH₃), 1.15 (s, 9H, C(CH₃)₃), 2.82-2.86 (m, 2H, CH₂), 3.45 (t, J=7.8 Hz, CHOC(CH₃)₃), 3.78 (s, 3H, OCH₃), 6.63 (d, J=2.7 Hz, Ar—H), 6.72 (dd, J=8.7 Hz, 2.7 Hz, Ar—H), 7.22 (d, J=8.4 Hz, Ar—H). ¹³C NMR δ157.49, 138.17, 132.99, 126.44, 113.80, 111.47, 80.84, 72.17, 55.15, 49.96, 44.05, 42.69, 38.68, 37.16, 31.14, 29.82, 28.68, 27.19, 26.30, 23.40, 11.49.

### Ent-(17β)-3-methoxyestra-1,3,5(10)-trien-17-ol

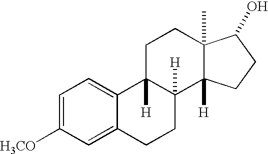

To a solution of ent-(17β)-17-(1,1-dimethylethoxy)-3-methoxyestra-1,3,5(10)-triene (0.3 g, 0.877 mmol) dissolved in 3 mL of anhydrous ethanol and 3 mL of THF, was added 3 mL of 6 N HCl. The mixture was heated with an oil bath (110° C.) for 2 h. The reaction was then cooled with an ice bath and 2.6 mL of 6 N NaOH was added until the solution was slightly acidic. After organic solvent removal, the residue was extracted into ethyl acetate. The combined extracts were washed with brine and dried over anhydrous sodium sulfate. Solvent removal gave a crude product (0.28 g) which was used directly for the preparation of ent-(17α)-17-iodo-3-methoxyestra-1,3,5(10)-triene.

### Ent-(17α)-17-iodo-3-methoxyestra-1,3,5(10)-triene

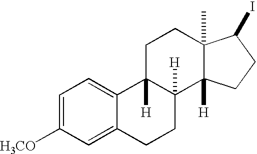

To a stirred solution of ent-(17β)-3-methoxyestra-1,3,5(10)-trien-17-ol (120 mg, 0.42 mmol) and triphenylphosphine (140 mg, 0.53 mmol) in 3 mL anhydrous toluene was added diethyl azodicarboxylate (140 mg, 0.80 mmol) followed by methyl iodide (130 mg, 0.92 mmol). A precipitate formed after the additions were complete. The reaction was stirred at room temperature for 0.5 h and then refluxed for 15 min. After the removal of solvent, a dark brown oil was obtained and the oil was purified by chromatography (silica gel eluted with 5% ethyl acetate in hexanes) to obtain the iodo compound (70 mg, 41% yield) and recovered steroid starting material (60 mg).

¹H NMR (CDCl₃) δ0.86 (s, 3H, CH₃), 2.84-2.89 (m, 2H, CH₂), 3.77 (s, 3H, OCH₃), 4.42 (d, 1H, CHI), 6.63 (d, 1H, J=1.8 Hz, Ar—H), 6.71 (dd, 1H, J=8.4 Hz, 2.4 Hz, Ar—H), 7.20 (d, 1H, J=8.4 Hz, Ar—H).

### Ent-3-methoxyestra-1,3,5(10)-triene

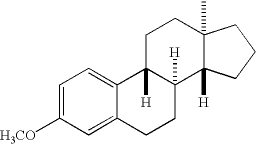

To a solution of ent-(17α)-17-iodo-3-methoxyestra-1,3,5(10)-triene (90 mg, 0.218 mmol) in anhydrous benzene (3 mL) under nitrogen were added 2,2′-azobis(2-methylpropionitrile) (13.8 mg, 0.084 mmol) and tributyltin hydride (0.3 mL). The reaction mixture was refluxed for 1.5 h. After solvent removal, the residue was purified by chromatography (silica gel eluted with 5% ethyl acetate in hexanes) to yield the product as an oil (70 mg).

hu 1H NMR (CDCl₃) δ0.74 (s, 3H, CH₃), 2.83-2.85 (m, 2H, CH₂), 3.77 (s, 3H, OCH₃), 6.63 (d, 1H, J=2.7 Hz, Ar—H), 6.71 (dd, 1H, J=8.7 Hz, 2.7 Hz, ArH), 7.22 (d, 1H. J=8.7 Hz, Ar—H).

### Ent-estra-1,3,5(10)-trien-3-ol

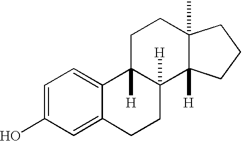

To a solution of 1.5 M diisobutylaluminum hydride (1.5 mL, 2.25 mmol) in toluene under nitrogen was added ent-3-methoxyestra-1,3,5(10)-triene (170 mg, 0.26 mmol) dissolved in 3 mL of anhydrous toluene. The reaction mixture was refluxed overnight, cooled to room temperature and ice was added. The reaction mixture was then acidified with 3 N HCl (3 mL) and extracted with ethyl acetate. The combined extracts were washed with brine and dried over anhydrous sodium sulfate. Removal of the solvent gave the crude product which was then purified by chromatography to yield the pure product (40 mg, 60% yield). After recrystallization from ethyl acetate-hexanes the product had: m.p, 130-131° C.; lit m.p. 134-135° C. [α]_{D}−100.5 (c=0.19, CDCl₃); lit [α]_{D}−92° C. (c=1, EtOH).

¹H NMR (CDCl₃) δ0.74 (s, 3H, CH₃), 2.80-2.81 (m, 2H, CH₂), 4.63 (s,OH), 6.56 (s, 1H, Ar—H), 6.63 (d, J=8.4 Hz, Ar—H), 7.17 (d, J=8.4 Hz, Ar—H). ¹³C NMR δ17.45, 20.47, 25.11, 26.69, 27.97, 29.68, 38.76, 39.08, 40.46, 41.00, 43.96, 53.51, 112.62, 115.26, 126.64, 133.33, 138.51, 153.27.

### EXAMPLE 5

### Preparation of 3′-Hydroxyspiro[cyclopentane-1,16′-estra[1,3,5(10),trien]-17′-one and (17β′)-Spiro[cyclopentane-1,16′-estra[1,3,5(10),trien]-3′,17′-one

The preparation of the above-referenced compounds is illustrated by the following reaction scheme. Details for the reactions carried out in each of the indicated steps, in order to prepare the various intermediate compounds and ultimately the above-referenced compounds, are provided below.

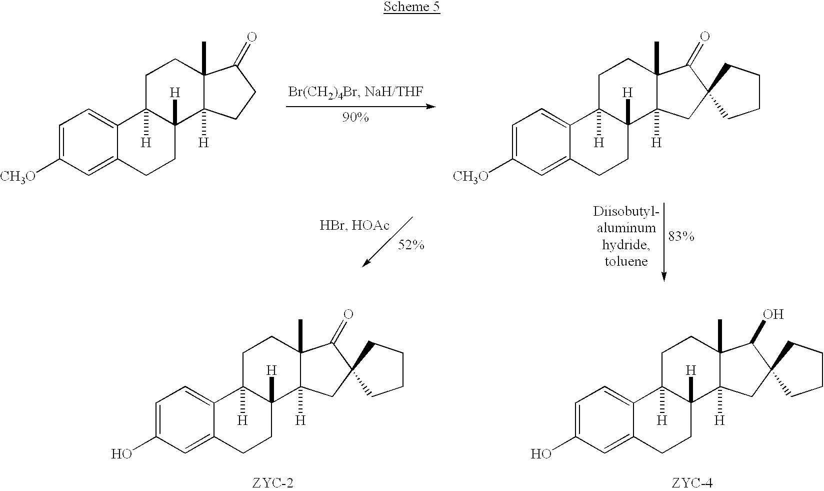

### 3′-Methoxyspiro[cyclopentane-1,16′-estra[1,3,5(10)]trien]-17′-one

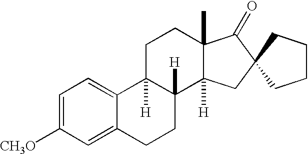

To a solution of 280 mg of 3-methoxyestrone in 7 mL anhydrous THF was added 260 mg of NaH and 1.2 mL of 1,4-dibromobutane. Then under nitrogen, the mixture was refluxed for 17 h. While cooling, 2 mL of ethyl alcohol was added slowly to destroy excess NaH. After gas evolution stopped, the mixture was poured into 50 g of ice. After the ice melted, the water was saturated with ammonium sulfate and extracted with diethyl ether (3×50 mL). The combined organic layers were dried with Na₂SO₄. After removing the solvent, 231 mg of crude yellow oily product was obtained. Purification by chromatography (silica gel eluted with 5% ethyl acetate in hexanes gave 0.3 g of pure compound in 90% yield. It had: m.p.122-123° C.; lit. m.p., 127-129° C.

¹H NMR (CDCl₃) δ0.95 (s, 3H, CH₃), 2.90 (m, 2H, CH₂), 3.80 (s, 3H, OCH₃), 6.60 (s, 1H, Ar—H), 6.72 (d, 1H, Ar—H), 7.22 (d,1H, Ar—H).

### 3′-Hydroxyspiro[cyclopentane-1,16′-estra[1,3,5(10)]trien]-17′-one

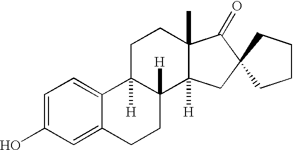

A mixture of 60 mg of 3′-methoxyspiro[cyclopentane-1,16′-estra[1,3,5(10)]trien]-17′-one (0.177 mmol) in 0.6 mL glacial acetic acid and 0.4 mL of 48% hydrobromic acid was heated at vigorous reflux under a nitrogen atmosphere. The solution became pale brown. After 40 min, a white solid precipitated. After 1 h, the reaction mixture was allowed to cool to room temperature and crushed ice was added. A pink solid was obtained. The pink solid was filtered, washed with water and dried in vacuum overnight at 50° C. The pink crude product (40 mg) was purified by chromatography (silica gel eluted with 20% ethyl acetate in hexanes) and 30 mg of pure compound was obtained (52% yield). The product was recrystallized from ethyl acetate and hexanes. The product (25.6 mg) had: m.p. 247-248° C.

1H NMR (CDCl₃) δ0.92 (s, 3H, CH₃), 2.84 (m, 2H, CH₂), 5.28 (s, 1H, OH), 6.60 (s, 1H, Ar—H), 6.70 (d, 1H, Ar—H), 7.18 (d, 1H, Ar—H).

### (17β′)-Spiro[cyclopentane-1,16′-estra[1,3,5(10)]triene]-3′,17′-diol

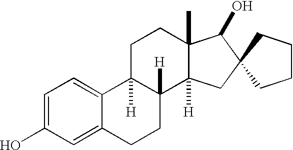

To a solution of 2 mL of 1.5 M diisobutyaluminun hydride (3 mmol), was added 90 mg of 3′-methoxyspiro[cyclopentane-1,16′-estra[1,3,5(10)]trien]-17′-one (0.266 mmol). Then the reaction mixture was refluxed 15 h under nitrogen. The reaction mixture was poured into 50 g of crushed ice. A white semi-solid that formed was dissolved by adding 2 N HCl (8 mL) and the reaction mixture was extracted with ethyl acetate (4×25 mL). The combined extracts were dried with anhyhdrous Na₂SO₄ and the solvent was removed. The crude product (0.1 g) was purified by chromatography (silica gel eluated with 20% ethyl acetate in hexanes. The pure product recrystallized from chloroform-hexanes (72 mg, 83% yield) and had: .m. p. 218-219° C.; lit. m.p. 229-230° C.

¹H NMR (CDCl₃) δ0.77 (s, 3H, CH₃), 2.82 (m, 2H, CH₂), 3.45 (m, 1H, CHOH), 4.62 (m, 1H, OH), 6.56 (s, 1H, Ar—H), 6.62 (d, J=8.4 Hz, Ar—H), 7.16 (d, J=8.4 Hz, Ar—H). ¹³C NMR (CDCl₃) δ11.7, 22.9, 24.5, 26.2, 27.2, 29.5, 34.9. 37.4, 38.1, 40.7, 42.3, 43.9, 47.3, 50.9, 88.4, 112.7, 115.2, 126.5, 132,.2, 137.7, 157.6, 177.3.

### EXAMPLE 6

### Comparative Study of Compound Activity

Method: HT-22 Cell Neuroprotection Assay

HT-22 cells (immortalized hippocampal neurons of murine origin) were maintained in DMEM media (Life Technologies, Inc., Gaitherburg, Md.) supplemented with 10% charcoal-stripped FBS (HyClone Laboratories, Inc., Logan, Utah) and 20 μg/mL gentamycin, according to standard culture conditions.

Cells were plated at a density of 5,000 cells/well in clear-bottomed Nunc 96-well plates (Fisher Scientific, Orlando, Fla.) and allowed to incubate overnight. Steroids dissolved in DMSO were added at concentrations ranging from 0.01-10 μM and were co-administered with glutamate (10 mM or 20 mM). DMSO was used at concentrations of 0.1% vol/vol as a vehicle control and had no discernible effect on cell viability. After about 16 h of glutamate exposure, cells were rinsed with PBS, pH 7.4, and viability was assessed by the addition of 25 μM calcein AM (Molecular Probes, Inc., Eugene, Oreg.) in PBS for 15 min at room temperature. Fluorescence was determined (excitation 485 nm, emission 530 nm) using a fluorescence FL600 microplate reader (Biotek, Winooski, Vt.). Cells that were lysed by addition of methanol were used for blank readings. All data were normalized to % cell death, as calculated by (control value −insult value)/control value×100.

Results

Test results for certain compounds of the present invention, some of which are described in the above Examples, along with known compounds (test for comparison or references purposes, including 17β-estradiol, ent-17β-estradiol, estrone and 17α-estradiol), are presented in Table 1, below.

TABLE 1
Neuroprotection of neuronal HT-22 cells against glutamate-
induced cell death by cytoprotective polycyclic compounds.

| | | Steroid concentration needed to | Steroid concentration needed to |
|---|---|---|---|
| | | protect 50% of neurons killed | protect 50% of neurons killed by |
| | | by 10 mM Glutamate | 20 mM Glutamate |
| | Compound | ED₅₀ (μM) | ED₅₀ (μM) |
| 1 | ZYC-13 | 0.23 | 1.00 |
| 2 | ZYC-28 | 0.46 | 0.56 |
| 3 | ZYC-10 | 0.47 | 0.93 |
| 4 | ZYC-12 | 0.53 | 1.43 |
| 5 | Ent-17β-Estradiol | 1.07 | 1.27 |
| 6 | ZYC-27 | 1.23 | 1.85 |
| 7 | ZYC-4 | 1.43 | 3.32 |
| 8 | ZYC-1 | 1.57 | 2.95 |
| 9 | 17β-Estradiol | 2.21 | 3.01 |
| 10 | Estrone | 3.03 | Not Determined |
| 11 | 17α-Estradiol | 3.10 | 16.12 |
| 12 | ZYC-2 | 4.21 | 10.16 |

In view of the above, it will be seen that the several objects of the invention are achieved. As various changes could be made in the above process and compounds without departing from the scope of the invention, it is intended that all matter contained in the provided description be interpreted as illustrative and not in a limiting sense.

What is claimed is:

1. A process for treating a cytodegenerative disease comprising administering to a subject in need thereof a compound having cytoprotective activity of the formula (I), or a diastereomer configuration thereof: 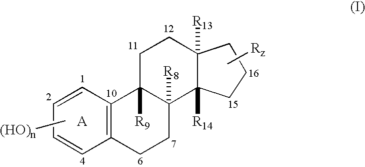
wherein
the compound optionally has one or more unsaturated bonds in conjugation with the aromatic A-ring between carbons 6 and 7, 8 and 9, or 9 and 11, in which event one or both of R⁸ and R⁹ will be absent;
n ranges from 1 to 4;
R⁸ and R⁹, when present, are independently hydrogen or alkyl;
R¹³ is hydrogen, substituted or unsubstituted hydrocarbyl, halo, amido, sulfate or nitrate;
R¹⁴ is hydrogen or alkyl;
R^{z} is hydrogen, hydroxy, oxo, substituted or unsubstituted hydrocarbyl, heterocycloalkyl, heterocycloalkenyl, halo, amido, sulfate, or nitrate; and,
carbon 17 and carbon 3 are not each hydroxy-substituted when (i) n is 1, (ii) the compound does not contain at least one unsaturated bond in conjugation with the aromatic A-ring, (iii) R⁸, R⁹ and R¹⁴ are hydrogen, and (iv) R¹³ is methyl.

2. The process of claim 1 wherein a carbon-carbon double bond is present in the compound between carbons 9 and 11.

3. The process of claim 2 wherein R⁸ and R¹⁴ are hydrogen and R¹³ is methyl.

4. The process of claim 3 wherein R^{z} is hydroxy.

5. The process of claim 4 wherein the compound is selected from: 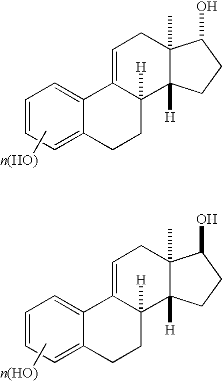
wherein n is as defined in claim 1.

6. The process of claim 1 wherein a carbon-carbon double bond is present in the compound between carbons 8 and 9.

7. The process of claim 6 wherein R¹⁴ is hydrogen and R¹³ is methyl.

8. The process of claim 7 wherein R^{z} is hydroxy.

9. The process of claim 8 wherein the compound is selected from: 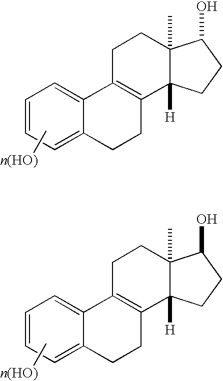
wherein n is as defined in claim 1.

10. The process of claim 1 wherein a carbon-carbon double bond is present in the compound between carbons 6 and 7.

11. The process of claim 10 wherein R⁸, R⁹ and R¹⁴ are hydrogen and R¹³ is methyl.

12. The process of claim 11 wherein R^{z} is hydroxy.

13. The process of claim 12 wherein the compound is selected from: 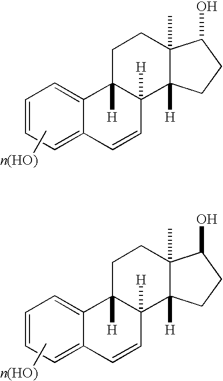
wherein n is as defined in claim 1.

14. The process of claim 1 wherein a carbon-carbon double bond is present in the compound between carbons 6 and 7 and 8 and 9.

15. The process of claim 14 wherein R¹⁴ is hydrogen and R¹³ is methyl.

16. The process of claim 15 wherein R^{z} is hydroxy.

17. The process of claim 16 wherein the compound is selected from: 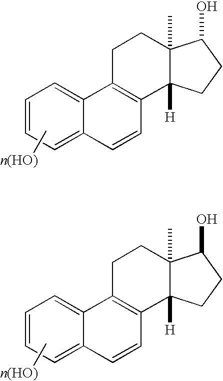
wherein n is as defined in claim 1.

18. The process of claim 1 wherein R⁸, R⁹ and R¹⁴ are hydrogen and R¹³ is methyl.

19. The process of claim 18 wherein R^{z} is hydrogen.

20. The process of claim 19 wherein the compound is: 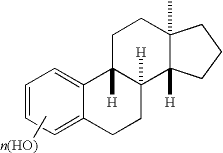
wherein n is as defined in claim 1.

21. The process of claim 18 wherein R^{z} is cycloalkyl or cycloalkenyl.

22. The process of claim 21 wherein R^{z} is a spiro structure, a carbon in the D-ring of the compound also being a carbon in the cyclic R^{z} substituent.

23. The process of claim 22 wherein the compound is: 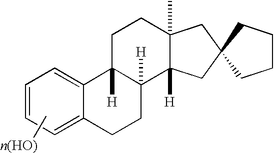
wherein n is as defined in claim 1.

24. The process of claim 23 wherein the D ring is additionally substituted with a hydroxy group or an oxo group.

25. The process of claim 24 wherein the compound is selected from: 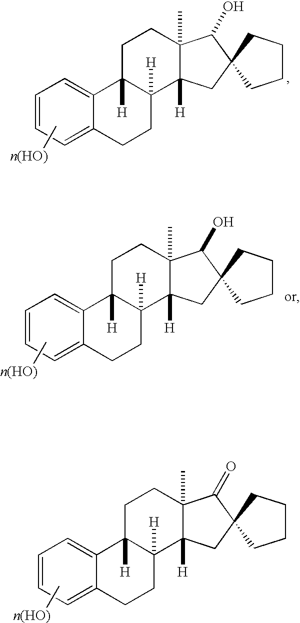
wherein n is as defined in claim 1.

26. The process of claim 1 comprising administering a pharmaceutical composition comprising said compound and a pharmaceutically acceptable carrier, excipient or diluent.

27. The process of claim 1 wherein said subject is an animal.

28. The process of claim 1 wherein said subject is a human.

29. A process for treating a cytodegenerative disease comprising administering to a subject in need thereof a compound having cytoprotective activity of formula (II), or a stereoisomeric configuration thereof: 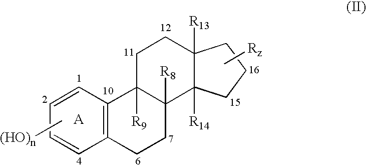
wherein
the compound optionally has one or more unsaturated bonds in conjugation with the aromatic A-ring between carbons 6 and 7, 8 and 9, or 9 and 11, in which event one or both of R⁸ and R⁹ will be absent;
n ranges from 1 to 4;
R⁸ and R⁹, when present, are independently hydrogen or alkyl;
R¹³ is hydrogen, substituted or unsubstituted hydrocarbyl, halo, amido, sulfate or nitrate;
R¹⁴ is hydrogen or alkyl;
R^{z} is substituted or unsubstituted cycloalkyl or cycloalkenyl, or substituted or unsubstituted heterocycloalkyl or heterocycloalkenyl.

30. The process of claim 29 wherein R^{z} is a spiro structure, a carbon in the D-ring of the compound also being a carbon in the cyclic Rz substituent.

31. The process of claim 30 wherein the compound is: 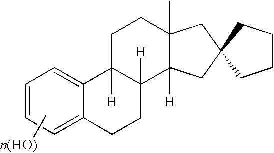
wherein n is defined in claim 29.

32. The process of claim 31 wherein the compound has the configuration R8α, R9β, R13α, and R14β.

33. The process of claim 31 wherein the compound has the configuration R8β, R9α, R13β, and R14α.

34. The process of claim 29 wherein the compound has the configuration R8α, R9β, R13α, and R14β.

35. The process of claim 29 wherein the compound has the configuration R8β, R9α, R13β, and R14α.

36. A compound having cytoprotective activity, the compound having the formula (I), or a diastereomeric configuration thereof: 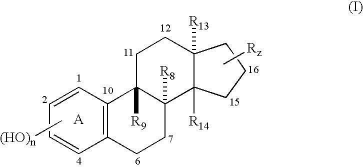
wherein
the compound optionally has one or more unsaturated bonds in conjugation with the aromatic A ring between carbons 6 and 7, 8 and 9, or 9 and 11, in which event one or both of R⁸ and R⁹ will be absent;
n ranges from 1 to 4;
R⁸ and R⁹, when present, are independently hydrogen or alkyl;
R¹³ is hydrogen, substituted or unsubstituted hydrocarbyl, halo, amido, sulfate or nitrate;
R¹⁴ is hydrogen or alkyl;
R^{z} is hydrogen, hydroxy, oxo, substituted or unsubstituted hydrocarbyl, heterocycloalkyl, heterocycloalkenyl, halo, amido, sulfate, or nitrate, provided however, when (i) the compound does not contain at least one unsaturated bond in conjugation with the aromatic A-ring, (ii) R⁸, R⁹ and R¹⁴ are hydrogen, and (iii) R¹³ is methyl, R^{z} is other than hydrogen and is not hydroxy or oxo when the D-ring is only substituted at carbon 17.

37. The compound of claim 36 wherein, when the compound has one of the following structures: 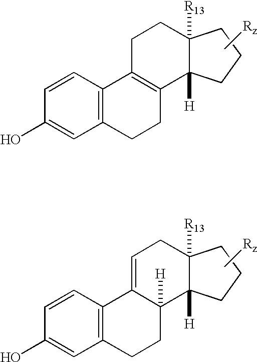
wherein R¹³ is methyl and R^{z} is other than hydroxy.

38. The compound of claim 37 wherein R^{z} is cycloalkyl or cycloalkenyl.
